(19) United States
(12) Patent Application Publication
(10) Pub. No.: US 2003/0181509 A1
(43) Pub. Date: Sep. 25, 2003

(54) Serotonin and catecholamine system segment optimization technology

(76) Inventor: Martin C. Hinz, Duluth, MN (US)

Correspondence Address: JOEL D. SKINNER, JR., SKINNER AND ASSOCIATES, 212 COMMERCIAL ST., HUDSON, WI, 54016, US

(21) Appl. No.: 10/394,597

(22) Filed: Mar. 21, 2003

Related U.S. Application Data
(60) Provisional application No. 60/366,983, filed on Mar. 21, 2002.

(51) Int. Cl. A61K 31/405; A61K 31/198

(52) U.S. Cl. 514/419; 514/567

(57) ABSTRACT

A method of treating neurotransmitter dysfunction in a patient. The method includes the step of administering an amino acid precursor of a catecholamine in a balanced and effective therapeutic range. The catecholamine precursor is preferably L-dopa, but may alternatively be tyrosine, D,L-Phenylalanine or an active isomer thereof, and N-acetyl-L-tyrosine or other amino acid precursor of L-dopa. An amino acid precursor of serotonin in an effective therapeutic range, is also administered. The serotonin precursor is preferably 5-HTP, but may alternatively be tryptophan. At least one cofactor is also preferably administered. Cofactor options include Vitamin B6, Vitamin C, Calcium, Folate, and Cysteine. A method of periodic administration and patient checking is also disclosed.

### CROSS-REFERENCE TO RELATED APPLICATIONS, IF ANY

This application claims the benefit under 35 U.S.C. §119(e) of co-pending U.S. Provisional Patent Application Serial No. 60/366,983, filed Mar. 21, 2002, which is hereby incorporated by reference.

### 37 C.F.R. §1.71(e) AUTHORIZATION

A portion of the disclosure of this patent document contains material which is subject to copyright protection. The copyright owner has no objection to the facsimile reproduction by anyone of the patent document or the patent disclosure, as it appears in the US Patent and Trademark Office patent file or records, but otherwise reserves all copyright rights whatsoever.

### STATEMENT REGARDING FEDERALLY SPONSORED RESEARCH OR DEVELOPMENT

Not applicable.

### REFERENCE TO A MICROFICHE APPENDIX, IF ANY

Not applicable.

### BACKGROUND

1. Field

The present invention relates, generally, to biomedical technology. More particularly, the invention relates to a technology for optimizing the serotonin and catecholamine systems. Most particularly, the invention relates to safe, effective compositions, methods and therapies for balancing, treating and optimizing the serotonin and catecholamine neurotransmitter systems in humans. The compositions, methods and techniques of the invention have broad applicability with respect to neurotransmitter dysfunction, including disease. The compositions, methods, and techniques may also be useful in other fields.

2. Background Information

The nervous system is the human body's key communications network. Along with the endocrine system, it provides most of the control functions of the body. The main parts of the nervous system are the brain, the spinal cord (which together with the brain makes up the central nervous system (CNS)), and the peripheral nervous system. The nerves are comprised of groups of neurons. Neurons transmit impulses or signals. Each neuron comprises a cell body or soma, dendrites that receive chemical signals from other neurons and axons that convey the signals as electrical impulses. A synapse is the junction point from one neuron to another. A great deal of signal control occurs at the synapse. In most chemical synapses, a first (pre-) neuron secretes a neurotransmitter into the synapse and this in turn acts on receptor proteins in the membrane of the next (post-) neuron. The transmitters may to excite the neuron, inhibit it, or modify its sensitivity in some other way.

Presently, 183 substances have been identified which can function as a neurotransmitter (synaptic transmitter) in the central nervous system. Master regulation of the neurotransmitters of the central nervous system neurotransmitters is attributed to the serotonin system and the catecholamine system.

Catecholamines include dopamine, norepinephrine and epinephrine. Both serotonin and catecholamines include relatively small molecules, which act relatively fast. These transmitters cause most of the acute responses of the nervous system, such as transmission of sensory signals to and inside the brain and motor signals back to the muscles. The catecholamines and serotonin are synthesized in the cytosol of presynaptic terminals. Presynaptic terminals are small knobs, which lie primarily on the surface of the dendrites. The synthesized transmitters are absorbed by transmitter vesicles in the terminals. Transmitters are released from the terminals by an action potential mechanism and cross a small synaptic cleft where they act on the post synaptic membrane receptors as discussed above. After a transmitter is released at a nerve ending, it is either destroyed or removed to prevent continued action. Removal mechanisms include diffusion of the transmitter out of the cleft, enzymatic destruction of the transmitter within the cleft itself, and transmitter re-uptake, which is the active transport back into the presynaptic terminal itself for reuse.

The catecholamine norepinephrine is secreted by may neurons whose cell bodies are located in the brain stem and hypothalamus. It is believed to help control overall activity and mood of the mind. Norepinephrine is also secreted by most of the post ganglionic mehnrons of the sympathetic (visceral functions of the body such as arterial blood pressure, gastrointestinal activity, urination, sweating and body temperature) nervous system, where it excites some organs and inhibits others. The catecholamine dopamine in the central nervous system is secreted by neurons that originate in the substantia nigra. The effect of dopamine is usually inhibition. Serotonin in the central nervous system is secreted by nuclei that originate in the median raphe of the brain stem. Serotonin acts as an inhibitor of pain pathways in the cord, and it is also believed to help control the mood and to regulate sleep through it role as a precursor of melatonin.

It is known from applicant's work that the serotonin system and the catecholamine system effectively work as one unit (hereinafter defined as The System). It is known from such work, that low levels of neurotransmitters are associated with numerous diseases and illnesses. Dysfunction (which includes disease or illness, dysfunction, sub-optimal performance of systems dependant on the neurotransmitters for regulation and function, or other malady relating to the catecholamine and/or serotonin neurotransmitter systems) results from suboptimal transfer of electrical energy between the input of the pre-synaptic neuron and output of post-synaptic neurons and/or neuron bundles of The System.

Dysfunction of the neurons of the central nervous system, in general, give rise to diseases and symptoms related to psychiatric illness and master control centers such as eating disorders, Parkinsonism, and the like. Dysfunction of neurons of the peripheral nervous system, in general, produces end organ disease, sub-optimal results, and dysfunction. The primary mechanism of dysfunction is a discrepancy between the electrical input to the neurons or neuron bundles and the output of the neurons or neuron bundles of The System. Anything that affects the electrical outflow of the neuron bundles to give a disproportion between the inflow and the outflow of electric energy can cause dysfunction. Examples of mechanisms and considerations of dysfunction include but are not limited to:

Nutritional deficiency

Increased metabolism secondary to drugs and substances

Hyperexcretion

Receptor regulation

System damage

Neurotransmitter levels lower than the threshold induce dysfunction

Referring to FIG. 1, which illustrates the synaptic model, and FIG. 2, which illustrates the electrical inflow and outflow of a neuron, a discussion of these concepts and implications is provided below to facilitate understanding of the forces affecting and inducing dysfunction.

Nutritional Deficiency is where low levels of nutrient intake required by The System for synthesis of neurotransmitters induces low Relative Neurotransmitter Levels (RNL) leading to dysfunction. Neurotransmitters facilitate transmission of electrical impulses between the pre-synaptic and post-synaptic neuron. Low RNL can cause dysfunction. FIG. 3 illustrates the primary nutrient deficiencies that affect the serotonin system. In the case of the catecholamine system, the critical nutrients are tyrosine (or its amino acid precursors), vitamin B6, vitamin C, and calcium; although the cysteine, methionine, S-adenosylmethionine (SAMe) and cortisol systems as discussed further below, systems must be managed properly as well. To apply FIG. 3 to the catecholamine system the basic synaptic model is the same and tyrosine is substituted for tryptophan in the model, L-dopa is substituted for 5-HTP in the model, dopamine is substituted for serotonin in the model and substitution of the appropriate cofactors for vitamin B3 and vitamin B6 are made. With regards to applying FIG. 3 to the catecholamine system all other considerations are the same with regards to secretion of neurotransmitters, reuptake of neurotransmitters, metabolism of neurotransmitters and the like. For this discussion we use the serotonin system as the working model for discussion. Nutritional deficiency is an important concept in understanding the picture as a whole of dysfunction. Serotonin synthesis is dependent on proper levels of amino acid precursors tryptophan or 5-hydroxytryptophan (hereafter referred to as “5-HTP”) with cofactors being available in the system. Catecholamine synthesis is dependent on proper levels of tyrosine (or amino acid precursors of tyrosine) or 3-Hydroxy-L-,t,tyrosine (hereafter referred to as “L-dopa”) with cofactors being available in the system. The methods taught by this invention are effective in addressing “Nutritional Deficiency”.

In accordance with the present invention, RNL is the level of neurotransmitters in The System that needs to be established in order for The System to be free of dysfunction which in some systems is higher than the normal level of neurotransmitters found in systems not subjected substances that alter neurotransmitter distribution and while on standard nutritional intake. RNL needs to be addressed in treating systems with dysfunction and not the “normal range” or “reference range” as reported in standard laboratory testing in order to optimally treat dysfunction.

Regarding increased metabolism of neurotransmitters secondary to drugs and substances, as was discussed above, neurotransmitters of The System are found primarily in “the store” also known as “the vesicles” of the pre-synaptic neurons or axon terminal. Neurotransmitters of The System are metabolized primarily by the Monoamine Oxidase system (MAO) and the catecholamine-O-methyltransferase system (COMT) as illustrated in FIG. 4. Applicant has surmised that when neurotransmitters are in the vesicles of the pre-synaptic neuron, they are safe and not exposed to metabolism by the enzymes of the MAO and COMT systems. Drugs that cause excretion of neurotransmitters from the vesicles into the synapse, such as amphetamines, cause increase metabolism of neurotransmitters by the MAO and COMT enzyme systems and depletion of neurotransmitters in The System, provided that increased intake of nutrients needed by The System to synthesize neurotransmitters is not provided for. Drugs such as reuptake inhibitors, which block reuptake of neurotransmitters into the pre-synaptic neuron, increase levels of neurotransmitters in the synapse and in the process expose more neurotransmitter molecules to the COMT system and accelerated metabolism. In general, any substance or event that causes redistribution of neurotransmitters out of the vesicles of the axon terminals can lead to increased metabolism of neurotransmitters by the MAO and COMT enzyme systems. In the process, increased metabolism of neurotransmitter occurs and if proper intake of nutrients needed by The System for synthesis of neurotransmitters is not provided for, the net effect is decrease levels of neurotransmitter molecules in The System as a whole. With this increased metabolism of neurotransmitters, there is a depletion of neurotransmitters leading to exacerbation of dysfunction. Increased metabolism of neurotransmitters is an important concept in understanding the picture as a whole of neurotransmitter dysfunction. The methods taught by this invention are effective in addressing “Increased metabolism secondary to drugs and substances”.

Hyperexcretion is a state whereby the kidneys through an unknown mechanism of action are excreting inappropriate amounts of neurotransmitters into the urine causing depletion of neurotransmitters of the system in general. Regarding hyperexcretion of neurotransmitters of the system, depletion of systemic neurotransmitters of the system correlates with increased incidents of dysfunction in the systems involved. In one study performed in the research leading up to this invention, 23.7% of human subjects were hyperexcreting neurotransmitters in samples obtained late in the afternoon (N=402). Hyperexcretion is an important concept in understanding the picture as a whole of neurotransmitter dysfunction. The methods taught by this invention are effective in addressing hyperexcretion.

Regarding receptor regulation, FIG. 5 illustrates the concept of neurotransmitter receptor regulation whereby the receptors of the post-synaptic neuron are not static on the surface of the neuron cell membrane. As receptors are down regulated, they retract into the cell membrane or in states of being less sensitive to neurotransmitter stimulation and in the process become less sensitive to the effects of neurotransmitters causing the RNL of neurotransmitters in the synapse needed to equalize the inflow and outflow of electrical energy between the pre-synaptic and post-synaptic neurons to increase in order for The System to be free of dysfunction. Down regulation of neurotransmitter receptors include but are not limited to chronic stimulation by neurotransmitters, as well as certain drugs and substances. Receptor response is enhanced by cyclical changes in the synaptic neurotransmitter levels and down regulated by a constant higher levels of neurotransmitters in the synapse. Receptor regulation is an important concept in understanding the picture as a whole of neurotransmitter dysfunction. The methods taught by this invention are effective in addressing receptor regulation.

Regarding system damage, in general, neurons do not function individually; they function instead as bundles of neurons. Nerve bundles are critical to maintaining heath and life, if life relied on a single neuron instead of bundles for regulation of bodily functions and the neuron became damaged and unable to function, life would be in critical trouble. FIG. 6(a) is an illustration showing a bundle of 1,000 neurons, each neuron conducting one nannowatts of electricity. Electrical units are for illustration purposes only and do not reflect the actual electrical energy levels involved. The illustration shows 1,000 nannowatts in and 1,000 nannowatts out of the neuron bundle. FIG. 6(b) shows the effects of damaging 500 neurons to the point of being non-functional (in apoptosis), 1,000 nannowatts in and 500 nannowatts out of the neuron bundle. Herein lies the problem and is a teaching of this invention; the net outflow of the neuron bundles must be above a certain threshold in order for The System to be free of dysfunction. If neurotransmitter levels in the synapse are too low, dysfunction develops. If enough neurons of the bundle are damaged and the net outflow of the bundles becomes low enough relative to the inflow of electrical energy dysfunction develops. Dysfunction caused by low levels of neurotransmitters in the synapse and damage to the neurons of the bundles looks the same from a clinical standpoint. From a clinical standpoint, the treatment considerations for system damage and low outflow of electric energy is the same with the exception of group dosing needs to overcome these effects to The System is greater and the RNL level needed to prevent dysfunction is higher than in states where there is no disproportion between the electrical input and output from system damage.

In addition to neurotoxic damage to The System, there are other forms of damage such as mechanical damage. Traumatic injury to The System can cause permanent damage of the neurons of the bundles and in the process, which from a clinical standpoint, look and act exactly as those systems that have been exposed to a neurotoxin.

Virtually all methods known in the prior art for treating neurotransmitter dysfunction of The System have the ability to deplete neurotransmitters in The System and in the process do harm to The System. Limited knowledge existed prior to this invention on the use of neurotransmitter levels in the treatment of dysfunction or damage. Amino acid therapy is known, but it's use has not been optimal and it has produced negative side effects. Balance, as it relates to administration of amino acid precursors of the serotonin and catecholamine systems is discussed below. The use of L-dopa, tyrosine, or other amino acid precursors of dopamine without proper balance of serotonin precursors being administered simultaneously cause nausea, headache, anxiety and feelings of uneasiness in patients. Use of 5-HTP or other amino acid precursors of serotonins without proper balance of dopamine precursors being administered simultaneously cause hypersomnolence, nausea, and distraction for mental acuity. Up to 70% of subjects taking amino acid dosing of one or the other system alone or not in proper balance with precursors of the other system can experience side effects. Increasing side effects increases the rate at which subjects stop treatment and in the process distracts greatly from optimal outcomes.

The invention teaches proper use, in balance, of amino acid precursors of the catecholamine system and serotonin system. With proper balance in administering amino acids of the two systems, two results occur:

1. optimal outcomes are observed that are not possible in treatment with unbalanced amino acid precursors of the catecholamine and/or serotonin system.

2. minimal side effects are seen with higher dosing of amino acids needed in treating all states of dysfunction, because the side effects of amino acid precursors of one system seen at higher dosing levels needed to control dysfunction cancel out and diminish the side effects of the other system.

For these and other reasons, a need exists for the present invention.

All US patents and patent applications, and all other published documents mentioned anywhere in this application are hereby incorporated by reference in their entirety.

### BRIEF SUMMARY

The present invention provides a neurotransmitter system segment optimization method and therapy which is practical, reliable, accurate and efficient, and which is believed to fulfill a need and to constitute an improvement over the background technology.

In a broad aspect, the invention provides a method of treating a patient comprising the steps of administering an amino acid precursor of a catecholamine; and administering an amino acid precursor of serotonin.

In a more specific aspect, the invention provides a method of treating neurotransmitter dysfunction in a patient comprising the steps of:

a. administering an amino acid precursor of a catecholamine in an effective therapeutic range, the catecholamine precursor being selected from the group of amino acids consisting of L-dopa, tyrosine, D,L-Phenylalanine or an active isomer thereof, and N-acetyl-L-tyrosine;

b. administering an amino acid precursor of serotonin in an effective therapeutic range, the serotonin precursor being selected from the group of amino acids consisting of 5-HTP and tryptophan; and

c. administering at least one cofactor, selected from the group of cofactors consisting of vitamin B6, Vitamin C, Calcium, Folate, and Cysteine.

In a still more specific aspect, the invention provides a method of treating dysfunction in the serotonin and catecholamine neurotransmitter system in a patient comprising the steps of:

a. administering, daily, a first combination of components for at least seven days, the first combination comprising:

i. an amino acid precursor, preferably L-dopa, of a catecholamine component, in an effective therapeutic amount of approximately 120 mg;

ii. an amino acid precursor, preferably 5-HTP, of serotonin component, in an effective therapeutic amount of approximately 300 mg; and

iii. a cofactor component preferably consisting of vitamin B6, Vitamin C, Calcium, and Folate;

a first half, with respect to quantity, of the first combination being administered approximately at a morning meal, and the second half, with respect to quantity, of the first combination being administered at least approximately five to six hours before bedtime in the evening;

b. on the seventh day after initiation of treatment, determining whether dysfunction in the patient has been controlled; and

c. if, in step b, dysfunction has not been controlled, administering, daily, a second combination of components comprising:

i. an amino acid precursor, preferably L-dopa, of a catecholamine component in an effective therapeutic amount of approximately 60 mg; and

ii. an amino acid precursor, preferably 5-HTP, of serotonin component in an effective therapeutic amount of approximately 300 mg;

the first combination being administered a first half, with respect to quantity, of the first combination being administered approximately at a morning meal, and the second half, with respect to quantity, of the first combination being administered at least 4 to 5 hours later;

the second combination being administered at least approximately five to six hours before bedtime in the evening.

The features, advantages, benefits and objects of the invention will become clear to those skilled in the art by reference to the following description, claims and drawings.

### BRIEF DESCRIPTION OF THE SEVERAL VIEWS OF THE DRAWING

FIG. 1 illustrates a synaptic model with respect to the serotonin system, the catecholamine system being similar.

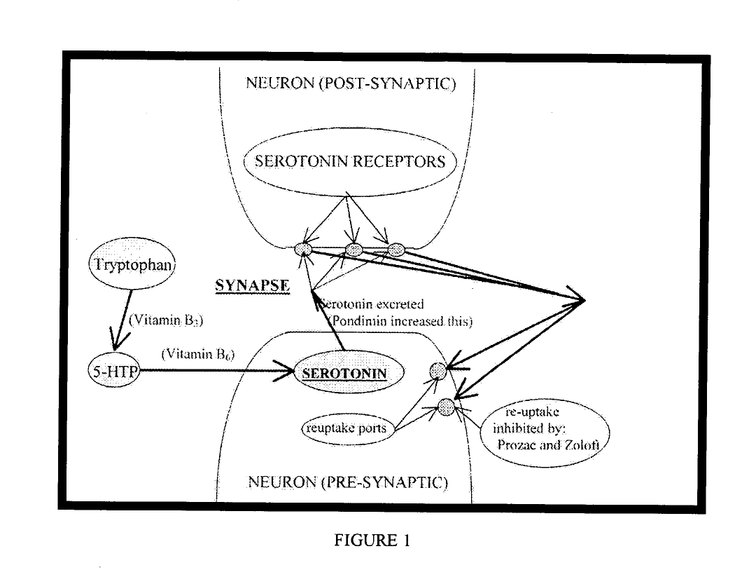

FIG. 2 illustrates the synaptic model showing electrical inflow and outflow.

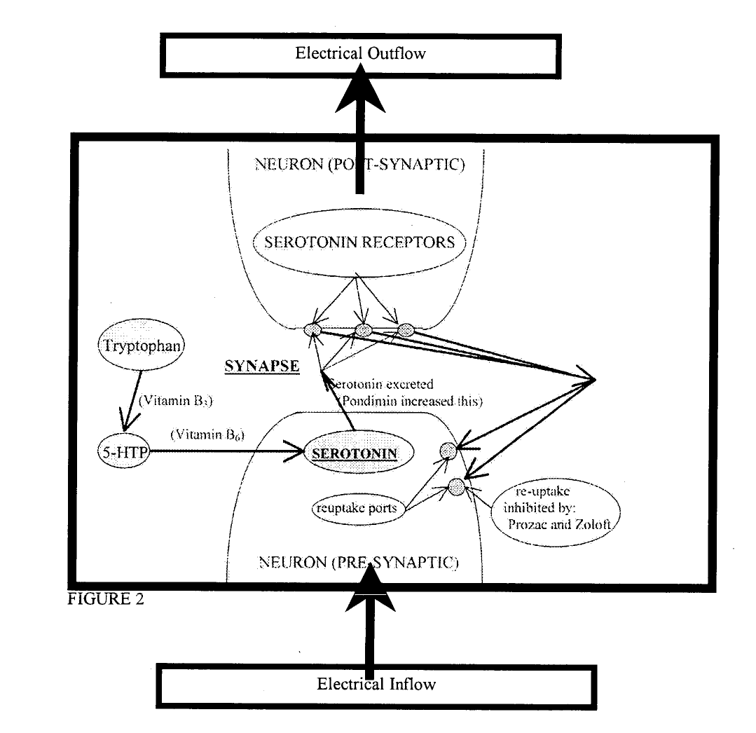

FIG. 3 illustrates the synaptic model showing nutritional deficiency occurring.

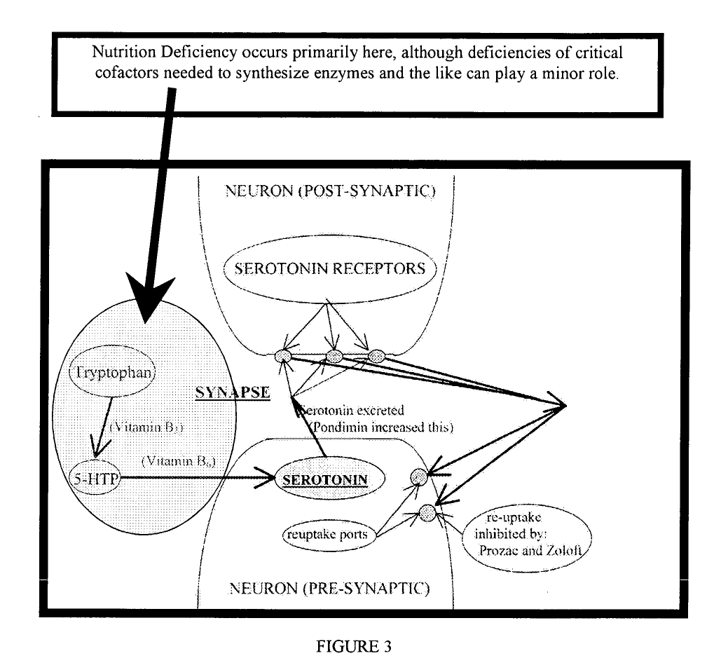

FIG. 4 illustrates the synaptic model showing metabolization of neurotransmitters by the Monoamine Oxidase System (MAO) and the catecholamine-O-methyltransferase system (COMT).

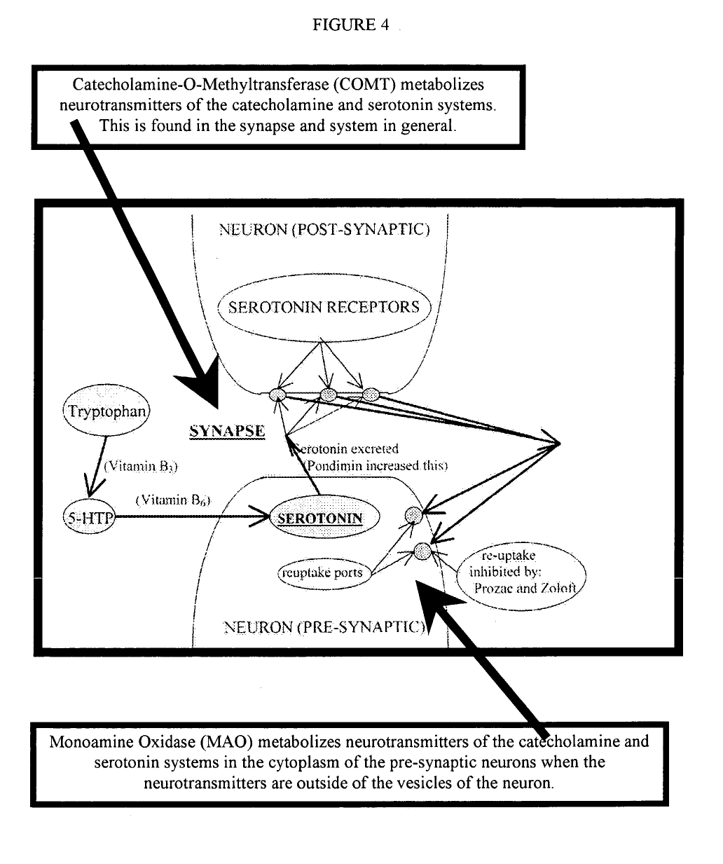

FIG. 5 illustrates receptor up-regulation and down-regulation.

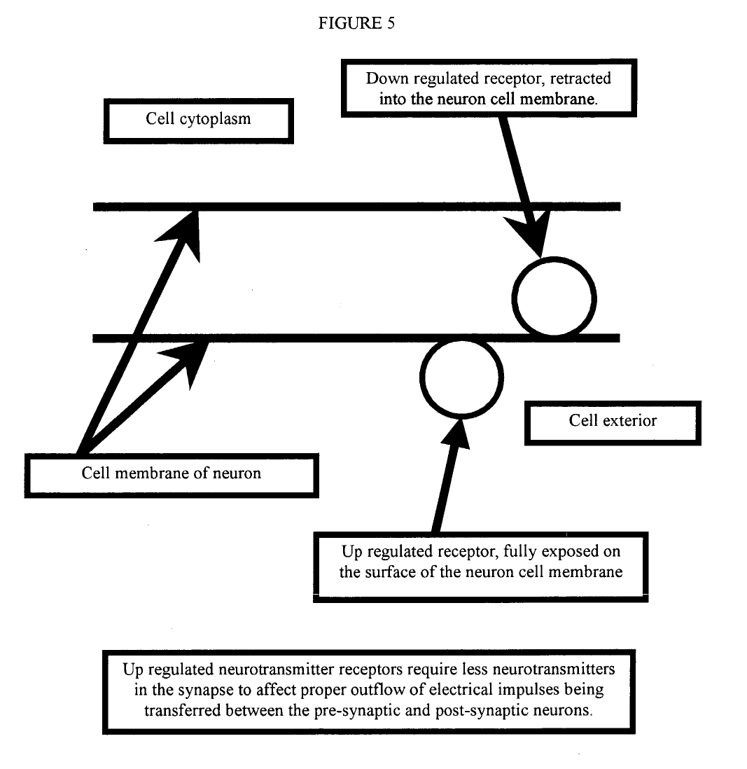

FIGS. 6A-C illustrate system damage, FIG. 6A depicting a normal system with 1000 nannowatts in, and 1000 nannowatts out (electrical units being for illustrative purposes only), FIG. 6B depicting a damaged system with half of the neurons in apoptosis and only 500 nannowatts out, and FIG. 6C depicting a damaged system compensated by establishing RNL above those normally found in the system and 1000 nannowatts out.

FIG. 7 illustrates RNL requirements to prevent dysfunction.

FIG. 8 illustrates relative system needs with respect to dopamine and serotonin, with the addition of L-dopa.

FIG. 9 illustrates balance of the catecholamine and serotonin systems.

### DETAILED DESCRIPTION

The embodiments of the invention described is intended to be illustrative and not to be exhaustive or limit the invention to the exact forms disclosed. The embodiments are chosen and described so that persons skilled in the art will be able to understand the invention and the manner and process of making and using it.

The teachings of this invention, in general, relate to optimizing group outcomes in the treatment of the neurotransmitter system (The System) in the management of dysfunction in human beings. However, the teachings may also be useful in any life form where the catecholamine system and the serotonin system is found, such as other animals. The invention provides the ability to optimize group results in the treatment of The System related dysfunction and a safe and effective method to gain control of The System in the treatment of dysfunction, as well as facilitate optimal function for systems dependant on the catecholamine and/or serotonin systems for regulation and function. Dosings listed in this description are for obtaining optimal results in a human population. Adjustment in dosing for non-human populations should be made based on body size and response.

1. General Discussion

The ability to differentiate systems with illness due to low levels of neurotransmitters in the synapse from those that have a component of damage came about through research wherein systems exposed to specific neurotoxic substances in the past required higher levels of neurotransmitters to compensate for dysfunction. In evaluation of the needs of a system to obtain the desired response in controlling dysfunction, it has been shown that subjects with a history of taking the neurotoxin fenfluramine (a component of a popularly known combination Phen-fen) with known neurotoxicity to The System needed to establish significantly higher neurotransmitter levels to obtain the desired response of controlling dysfunction in comparison to those that have no history of fenfluramine exposure. Other neurotoxins include but not limited to are amphetamine, 3,4-methylenedioxy-methamphetamine heavy metals, pesticides, certain drugs and a host of other substances.

Damage to neurons that is mechanically related and permanent, from a functional standpoint, requires establishing neurotransmitter levels higher than normally found in The System without treatment in order to compensate for neurotransmitter dysfunction through hyperstimulation of the remaining viable neurons.

In order to obtain the proper response of dysfunction being under control in those systems who have been exposed to neurotoxins in the past and/or have suffered system damage of other etiology, neurotransmitter levels higher than normal must be established in The System thereby hyperexciting the remaining non-damaged neurons and receptors to increase the output of the remaining viable neurons in the bundles.

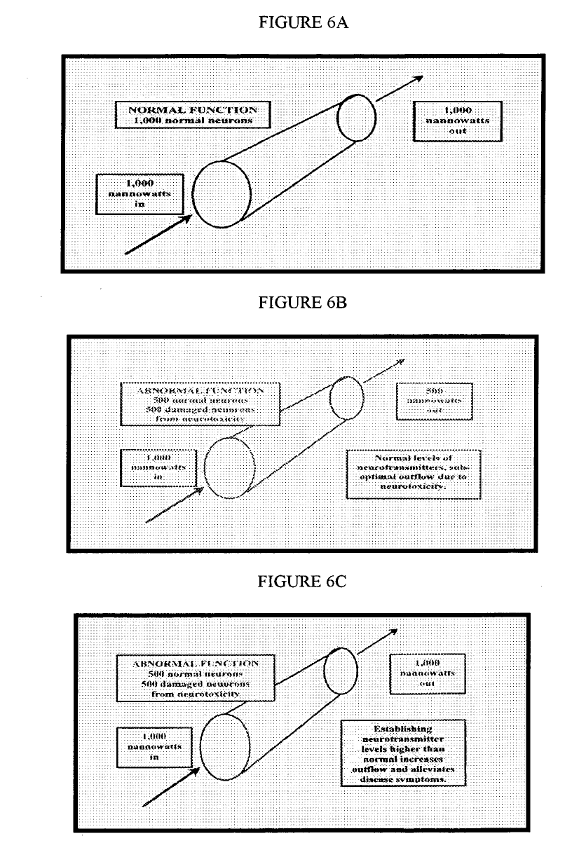

FIG. 6(c) shows how, in order to give a symptom free state in The System suffering from damage, hyperexcitement of the remaining viable neurons of the neuron bundles must take place by establishing neurotransmitter electrical output levels that are higher than normal. Half of the neurons are illustrated as being damaged to a state of apoptosis. With proper treatment, the electric output level of remaining viable neurotransmitters in the synapse is increased above normal levels causing the electrical outflow of the neuron bundle to rise above the threshold needed to keep The System symptom free of disease, this is discussed more in the following. The methods taught by this invention are effective in addressing “System damage”.

It is known that, “Drugs that work with neurotransmitters do not work if there is not enough neurotransmitters to work with.” Applicant has recognized that drugs that work with neurotransmitters of one system may not produce the desired effects if the neurotransmitter levels of the other system are too low. This supports the assertion that both systems must be functioning properly for The System to function normally and proper response to stimuli to be observed. Examples of this are discussed below.

In general, neurons do not function as a single neuron, they function as “bundles of neurons” made up of multiple neurons functioning as a unit. Optimal function of these neuron bundles depends on the proper flow of electrical energy through the bundle. If the electrical energy out of the neuron bundle is decreased enough relative to the amount of electrical energy going into the neuron bundle by various mechanisms discussed below, dysfunction will occur. Neurotransmitter levels lower than the threshold induce dysfunction. Based on clinical observations and database research of the applicant, there exists a threshold of electrical energy output of the neuron bundles above which facilitates neurotransmitter function and below which there is dysfunction is present. The model of “Increased metabolism secondary to drugs and substances” is one example of a mechanism of action for neurotransmitter depletion that many drop below the threshold. The threshold on the serotonin side of The System is like a common light switch; it is either on or off. From an observed outcomes standpoint, dysfunction is either present or not as serotonin neurotransmitter concentration levels in the synapse rises and falls, above and below the threshold needed to keep The System free of dysfunction. An example of this is with depression where the subject has responded to treatment with a highly selective serotonin reuptake inhibitor such as Citalopram and is no longer suffering the symptoms of dysfunction associated with depression. After several months of treatment the subject literally awakes one day to find that the symptoms of dysfunction in the form of depression have returned. It is at that point in time that the neurotransmitter levels have dropped below the threshold needed to keep The System free of dysfunction just like a light switch turning off.

As a tachyphylaxis develops due to serotonin levels dropping below the threshold needed to prevent dysfunction, it is like a light switch with the symptoms associated with dysfunction being “on or off”. The catecholamine system is more like a dimmer switch on a light where the desired response from the drug, substance, or compound slowly fades out over time to a full tachyphylaxis. A reason for developing a sub-optimal response such as this that gradually diminishes can be due to the COMT and MAO systems causing metabolism that is unbalanced with synthesis. Display of the threshold from a clinical standpoint is identifiable. A subject is doing well and the dysfunction you are treating is under control then the subject misses one or two doses of amino acids, drugs, or other substances that affect distribution of the neurotransmitters in The System and the dysfunction returns. At that point The System neurotransmitter levels drop below the threshold needed to keep The System free of dysfunction. Cases were observed where the dosing of amino acids, drugs, or other substance was just above the threshold and by lowering the daily dosing a very small amount; it caused the dysfunction to return. The awareness of the threshold and its importance in treating systems explains why systems in group treatment need such a diverse range of amino acids or drug dosing from subject to subject in order to obtain control of dysfunction.

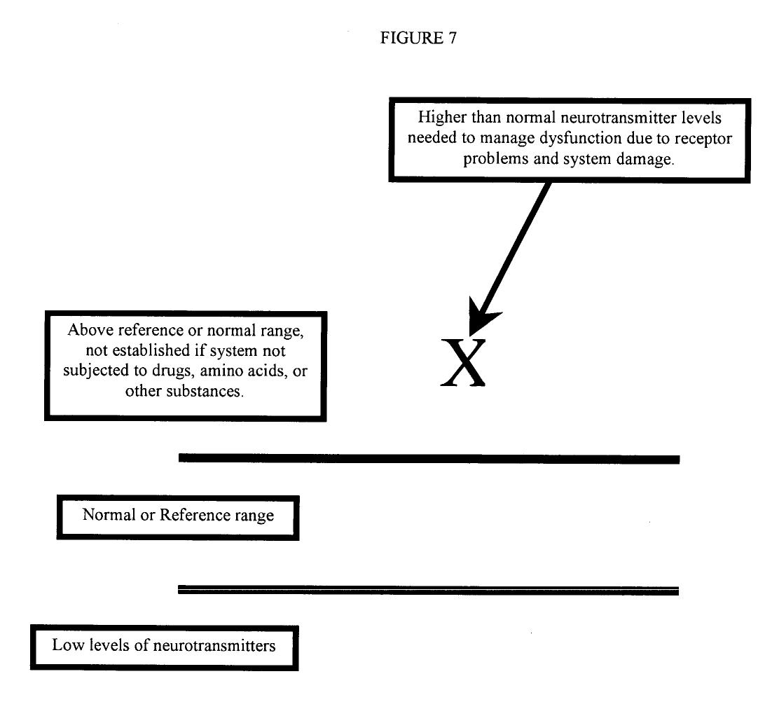

Nutritional deficiency, increased metabolism secondary to drugs and substances, and hyperexcretion related dysfunction can be managed by establishing neurotransmitter levels in The System that are within the normal range as commonly defined by laboratory testing. Whereas receptor considerations and system damage increases the RNL needed to keep dysfunction under control as noted in FIG. 7. By increasing the neurotransmitter levels to levels higher than normally found in systems with normal amino acid intake of a diet that is normal for The System, a state of hyperexcitement exists whereby the remaining viable receptors in the damaged bundle are stimulated above normal levels to give a relatively normal and properly functioning electrical outflow of the neuron bundles thus controlling the dysfunction.

In any system there can be a mixture of causes for dysfunction. Individual mechanisms of action may correlate more with certain disease states and states of dysfunction than others. For example, it was found that hyperexcretion of neurotransmitters not only correlated with increased incidence of dysfunction in general, it also correlated specifically with decreased cognitive function such as Attention Deficit Hyperactivity Disorder (ADHD), dementia in the elderly, etc. System levels may need to be increased higher than normal ranges to control dysfunction from system damage, receptor damage, receptor regulation and other considerations relating to compromised electrical outflow in the presence of normal neurotransmitter levels.

In the broadest sense, the teaching of the invention involves the use of L-dopa and 5-HTP to optimize the catecholamine system and serotonin system. Tyrosine and tryptophan (or other amino acid precursors of dopamine and serotonin, respectively) may be substituted for L-dopa and 5-HTP respectively with more limited results in the when suboptimal electrical outflow is present in the presence of normal neurotransmitter levels. The invention involves optimizing both systems in support of applications involving The System. The ratio of L-dopa to 5-HTP for optimal results is generally 1:3 on a milligram for milligram basis and the ratio of tyrosine to 5-HTP for optimal results when used is 10:1.The ratio of Phenylalanine to 5-HTP for optimal results, when used, is 10:1, and the ratio of N-acetyl-tyrosine to 5-HTP for optimal results when used is 5:1. Other amino acid precursors of dopamine and serotonin may be used with considerations for ratios to obtain optimal results, but if the goal is optimal results with group treatment ratios close (within 85%) to these should be used. Proper use of amino acid ratios is hereafter referred to as “balanced”.

The inflow of electrical energy and the outflow of electrical energy into and out of neurons and neuron bundles are analogous to a computer program. With a computer program there is input into a programmed segment, which is manipulated to affect an output. The computer program can be a “segment” or “step” within another computer program performing a specific function. In turn, the computer program segment may be useful if the segment it represents causes the other computer program that it functions in to work new or better. With the serotonin/catecholamine segment (i.e. The System) the electrical energy input is manipulated by the status of the neurons or neuron bundles to affect an output. Just as a computer program which can make other programs work in a new way or better when integrated into the other program, optimizing the serotonin/catecholamine segment as taught herein will cause other systems that interact with the system to work in a new way or better.

When 5-HTP is introduced into The System, it is synthesized freely into serotonin (5-HT) without biochemical regulation affecting the amount of serotonin that can be synthesized. This gives 5-HTP the ability to establish serotonin levels that are higher than normally found in the body than by ingestion of the precursor tryptophan, which is subject to biochemical regulation with regards to synthesis. It is noted that 5-HTP is not found in normal diets in significant amounts. Normally production of serotonin relies primarily on tryptophan in systems on normal dietary amino acid intake, this is biochemically regulated by the “5-HTP/tryptophan hydroxylase” feed back loop and limits serotonin synthesis in the normal system above the given normal range.

Normally, The System relies primarily on tyrosine (or amino acid precursors of tyrosine to synthesize tyrosine) intake from dietary means, which is synthesized into L-dopa via the enzyme tyrosine hydroxylase, to synthesize catecholamines. In the normal system, the conversion of tyrosine to L-dopa is regulated by the “norepinephrine/tyrosine hydroxylase” biochemical regulatory feed back loop, which regulates formation of the catecholamines dopamine, norepinephrine, and epinephrine above the given normal range. L-dopa, not being subject to a biochemical regulatory mechanism, has the ability to establish catecholamine levels that are higher than normal in The System. It is noted that L-dopa is not normally found in diets of normal systems in significant amounts.

It is known that the amino acids L-dopa and 5-HTP as well as the cofactors required in the synthesis of neurotransmitters are water-soluble and freely cross the blood brain barrier. The neurotransmitters dopamine, norepinephrine, epinephrine and serotonin are fat-soluble and do not cross the blood brain barrier. It is a teaching of this invention that due to the fact that L-dopa and 5-HTP are water-soluble and distribute freely and equally throughout the body including the central nervous system and the peripheral system as time passes, an equilibrium occurs between the central nervous system and peripheral system where the precursors of neurotransmitters, namely L-dopa and 5-HTP, that are turned freely into neurotransmitters without being subject to biochemical enzyme regulatory mechanisms, are in equilibrium. At this point, a true correlation exists between the central and peripheral neurotransmitter levels. Time to equilibrium with steady state intake of amino acids and other nutrients in general is five to seven days.

The dosing of amino acids needed to control dysfunction vary widely in group system treatment, for example dosing needed to control dysfunction on the high end needs may be 15 to 20 times the dosing of amino acids needed to control dysfunction on the low end needs for a group. The goal of the invention is to optimize group outcomes in treatment of dysfunction of The System.

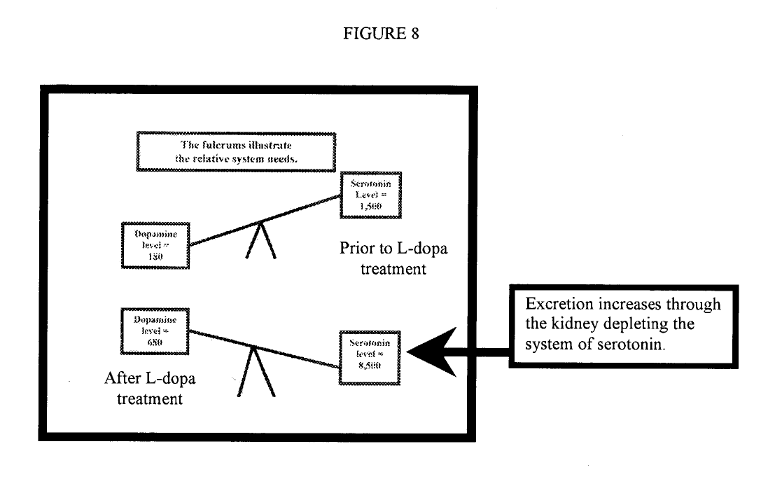

FIG. 8 shows urinary neurotransmitter testing results in subjects taking a fixed dose of 5-HTP and tyrosine to which L-dopa was added in the amount of 360 mg per day. The units reported are for urinary testing in “micrograms of neurotransmitter per gram of creatinine”. The second testing date was after the L-dopa was started with no change in other amino acid dosing. The laboratory performing the testing defined the reference ranges as follows:

1. Dopamine 65-250 micrograms per gram of creatinine

2. Norepinephrine 20-45 micrograms per gram of creatinine

3. Epinephrine 5-13 micrograms per gram of creatinine

4. Serotonin 75-250 micrograms per gram of creatinine

Of note is the fact that reported levels of dopamine, norepinephrine and serotonin are well above the normal range in the second set of tests which was conducted after starting L-dopa 360 mg per day and the epinephrine levels are in the upper end of the normal range.

| | | | | |
|---|---|---|---|---|
| DATE COLLECTED | Dopamine AM | Norepinephrine AM | Epinephrine AM | Serotonin AM |

INDIVIDUAL #1

| | | | | |
|---|---|---|---|---|
| Jan. 11, 2002 | 195.60 | 34.40 | 6.80 | 1169.20 |
| Jan. 18, 2002 | 496.40 | 57.20 | 9.20 | 8000.00 |

INDIVIDUAL #2

| | | | | |
|---|---|---|---|---|
| Oct. 31, 2001 | 154.00 | 25.60 | 3.20 | 453.50 |
| Jan. 13, 2002 | 334.30 | 80.90 | 9.60 | 2678.90 |

In individuals #1, #2, and as shown in FIG. 8, the only thing changed in the system was addition of L-dopa. In doing so, the urinary excretion of serotonin increased markedly causing the systemic levels of serotonin to decrease as the urinary excretion of serotonin increased. From research conducted, this illustrates vividly the teaching of the invention that changes affecting one system affect the other system.

FIG. 8 illustrates that changes to one system affects the other system. The square boxes of the fulcrums represent the systemic neurotransmitter levels and numbers in the boxes represent the urinary neurotransmitter levels. It is a teaching of this invention that the catecholamine system and the serotonin system functions essentially as one system and change cannot be made to the components of either without affecting the other system. The body synthesizes dopamine from L-dopa. Dopamine is synthesized to norepinephrine, and norepinephrine in turn is synthesized to epinephrine.

L-dopa→dopamine→norepinephrine→epinephrine

In two sets of urinary neurotransmitter testing in FIG. 8 from subjects #1 and #2, each contains two neurotransmitters tests. The earliest date of each set was performed without L-dopa being ingested into The System. The later date (the bottom test) of each set was performed after the subject had only added daily L-dopa to the ingestion with no other nutritional intake changes at the rate of 360 mg each day between tests. As noted in the bottom test of each set the L-dopa administration caused significant changes in the catecholamine system. In examining the serotonin levels, it is quite apparent that the serotonin system has also been affected even though no changes were made directly to The System.

As noted in previously 5-HTP is synthesized freely into serotonin without being subjected to a biochemical enzyme regulatory feed back loop. L-dopa, in the same manner, synthesized freely into dopamine within the body by the liver and neurons without being subjected to a biochemical enzyme regulatory loop feed back. These biochemical properties as they exist allow for theoretically unlimited and unregulated production of dopamine from L-dopa and serotonin from 5-HTP if proper levels of functioning associated enzymes and cofactors are found in The System.

The subjects in FIG. 8 were previously started on 5-HTP and tyrosine in an amount that caused urinary neurotransmitter testing to reveal serotonin levels that were approximately two to four times above the high end of the normal or reference range as reported by the laboratory. At which point the subject was in a state of hyperserotoninemia as evidenced by the laboratory testing. 5-HTP and tyrosine daily intakes by the subjects were continued at the same dosing to affect conditions as existed at the time the first test was carried out. The results show that administration of 360 mg per day of L-dopa caused a six to seven fold increase in urinary serotonin levels.

The invention teaches that as the catecholamine levels rise in the systems tested in FIG. 8, kidney excretion of catecholamines rise above the normal range as the body is taking steps to excrete catecholamines. Systemic levels, including inter-synaptic levels catecholamines, rise above the normal or reference range levels as evidenced by laboratory testing and outcomes are observed to include increased excretion of serotonin by the kidney. Catecholamine systems and the serotonin systems function as one system in balance. As the neurotransmitter levels of the catecholamine system rise the serotonin needs of the system decrease and The System compensates for this by increased excretion of serotonin. The same is true with the serotonin system. With the administration of 5-HTP, a point is reached where the inter-synaptic serotonin levels are optimal and The System begins to take steps to metabolize and excrete excess serotonin now found in The System as evidenced by increased urinary serotonin levels. Thus, by increasing the catecholamine levels of a system, the serotonin needs of The System decrease and by increasing the serotonin levels of a system the catecholamine needs of the system decrease.

System balance is illustrated in FIG. 8. As catecholamine levels rise in The System, the amount of serotonin in The System decreases secondary to excretion of serotonin, even though the amount of 5-HTP or other serotonin precursors taken into The System remains the same.

It is a teaching of this invention that Disease and illness, as well as drugs, substances, and compounds that affect primarily one system also affects the other neurotransmitter system. An example of this is drugs that cause excretion of neurotransmitters from the neurons of one system will lead to increased excretion of neurotransmitters of the other system in the urine and potential cause depletion of neurotransmitters of the other system.

It has been observed in clinical situations where subjects have extremely low levels of neurotransmitters in one system with higher than normal urinary neurotransmitter levels in the other system, that no response is seen from drugs that exert their effects on the higher system, or that increased dosing of the drug was needed to effectuate the desired clinical response. Under these same circumstances sub-optimal response to the drug may be seen.

Only to a certain point can markedly increased levels of neurotransmitters in one system compensate for low levels of neurotransmitters in the other system and if the neurotransmitter levels in the other system are too low, no response from the increased system will be seen no matter how high the levels of neurotransmitters are in that system.

The approach of using amino acid precursors that are precursors of only one of the systems or not balanced properly is not optimal since it does not facilitate both systems functioning optimally and in long-term treatment can lead to depletion of neurotransmitters of The System if no additional precursors of the other system are provided for. An example of this is shown in FIG. 8.

Applications of the invention relate to any application where any of neurotransmitter components of The System are involved. They include but are not limited to:

1. Treatment in and of itself in subjects suffering from central nervous system neurotransmitter diseases such as:

Depression

Anxiety

Panic attacks

Migraine headaches

Obesity

Bulimia

Anorexia

Premenstrual syndrome

Menopause

Insomnia

Hyperactivity

Attention deficit disorder

Impulsivity, obsessionality

Aggression

Inappropriate anger

Psychotic illness

Obsessive compulsive disorder

Fibromyalgia

Chronic fatigue syndrome

Chronic pain states

Adrenal fatigue

Attention Deficit Hyperactivity disorder

Parkinsonism

States of decreased cognitive function such as:

Dementia

Alzheimer's disease

2. Optimizing or enhancing the response in the system from drugs, substances, or compounds that produce their effects by interaction with neurotransmitters of The System. Any drug, substance, or compound that redistributes neurotransmitters of the catecholamine system or the serotonin system from the safety of the pre-synaptic vesicles to a place outside the vesicle where they come in contact with the COMT and MAO systems will benefit. Substances that exert their action by redistribution of neurotransmitters include but are not limited to:

Selective serotonin reuptake inhibitors (SSRI)

Citalopram

Fluvoxamine

Floxetine

Sertraline

Paroxetine

Hypericum

Serotonin norepinephrine reuptake inhibitors

Venlafaxine

Sibutramine

Other inhibitors of the catecholamine serotonin system

Bupropion

Excretors of neurotransmitters to include norepinephrine and/or serotonin

Amphetamines

Phentermine

Phendimetrazine

Benzphetamine

Diethyproprion

Caffeine

Ephedra (ephedrine)

Phenylpropanolamine

Combinations of these drugs such as “caffeine and ephedra”.

3. Reestablishing a response from a drug, substance, or compound that works with neurotransmitters and has developed a tachyphylaxis. As discussed further, drugs, substances and compounds that work by redistributing neurotransmitters levels from pre-synaptic vesicles to outside the vesicles where they come in contact with the COMT and MAO systems, thereby increasing metabolism of neurotransmitters, lead to the development of tachyphylaxis as the level of neurotransmitters in The System drop below the threshold needed to keep drugs that work with neurotransmitters functioning. The therapy of invention can reverse this process with optimal results.

4. The invention has the ability to establish a response in circumstances where no response is seen in systems when there has been an initiation of the drug, substance, or compound that is dependant on the catecholamine system and/or serotonin system for response.

5. The invention has the ability to illicit a display of new properties, as cited in the use and administration of the combination L-dopa and 5-HTP with a selective serotonin reuptake inhibitor or serotonin norepinephrine reuptake inhibitor (with the preferred drug being any compound containing the active isomer of Citalopram). It can induce a caliber of appetite suppression not known with the use of the individual components or combinations of 2 of the components.

6. Facilitating optimal innervations and regulation of systemic functions dependent on neurotransmitters of The System for such innervations and regulation. Examples of this include but not limited to:

a. Hormone dysfunction is not optimal until the neurotransmitters controlling the system are optimized. Dysfunction of the neurotransmitters of The System contributes greatly to hormone problems. Optimal results can be obtained in addressing systems innervated by The System. First, the neurotransmitters of the system are optimized first. For example, it was found that if hormone replacement therapy was administered to subjects prior to optimization of The System and The System was then optimized, on re-evaluation of the hormone system, it was found that hormone replacement in general was affected to the point where it had to be once again addressed and in some cases was no longer needed. Optimizing the neurotransmitters of The System prior to correcting problems of with systems innervated by The System is preferred.

b. Optimizing neurotransmitter levels in the GI tract to facilitate optimal function and treatment of disease.

Use of the combination L-dopa and 5-HTP (or other amino acid precursors of dopamine and serotonin) in proper ratios hereafter known as “balanced” is effective in reestablishing a clinical response from a drug, substance, or compound that is dependent on neurotransmitters to produce effects that has developed a tachyphylaxis. It is known that, drugs, substances and compounds that work with neurotransmitters do not work if there is not enough neurotransmitters to work with (i.e. they are dependant on neurotransmitters for the response observed). It is a teaching of the balanced elevation of the neurotransmitters in support of applications that substances that redistribute neurotransmitters from pre-synaptic vesicles of the axon terminal to the synapse and system lead to depletion of the neurotransmitters if proper dosing and ratios of amino acid precursor intake with cofactors is not provided for. L-dopa and 5-HTP both can function as amino acid precursors of the catecholamine system and the serotonin system respectively thereby preventing the tachyphylaxis if administered properly in accordance with the invention.

The Catecholamine-O-methyltransferase system (COMT) is found in the synapse or system, as a whole. They are the enzyme systems responsible for metabolizing neurotransmitters of The System. The Monoamine Oxidase system (MAO) is found in the cytoplasm of the pre-synaptic neurons outside of the vesicles. The MAO system is an enzyme system responsible for metabolizing neurotransmitters of The System within the pre-synaptic neurons. The COMT and the MAO systems are the systems within the body that are responsible for metabolism of neurotransmitters of the catecholamine and serotonin systems. By setting up conditions with the use of drugs, substances, and compounds that are dependent on neurotransmitters for their effects whereby neurotransmitters molecules are redistributed from the safety of the vesicles in the axon terminal (pre-synaptic neuron), increased metabolism of neurotransmitters occur leading to depletion of systemic levels of neurotransmitters if proper intake of neurotransmitter precursors and cofactors is not provided for. With such increased metabolism of neurotransmitters, if increased nutrients in the form of amino acid precursors and cofactors are not provided for so that synthesis is balanced with increased metabolism, a depletion of neurotransmitters can take place. The net result of this depletion is that sub-optimal results or tachyphylaxis is seen with the use of drugs, substances, or compounds that are dependent on neurotransmitters for their effects. When the drug, substance, or compound is no longer giving the desired or optimal response, the number of neurotransmitter molecules in The System has just dropped below threshold levels needed for the drug, substance or compound to continue functioning optimally or simply functioning at all. The balanced combination L-dopa with 5-HTP, with adequate levels of cofactors, provided by this invention, effectively increases the neurotransmitter levels in the catecholamine system and the serotonin system effectively resolving tachyphylaxis in the use of drugs, substances, and compounds that are dependent on neurotransmitters for their effects on a long-term basis.

The balanced combination of L-dopa and 5-HTP, with adequate levels of cofactors (“the balanced combination”) is effective in “establishing a clinical response in circumstances where no clinical response is seen in subjects from initiation of the drug, substance, or compound that is dependent on neurotransmitters for their effects”. It is known in literature that, “drugs, substances, or compounds that work with neurotransmitters do not work if there is not enough neurotransmitters to work with.” When no clinical response is seen to a drug, substance or compound that works with The System, the primary cause is neurotransmitter depletion in The System at the time of initiation of the use of the drug, substance, or compound that is dependent on neurotransmitter for their effects. It effectively increases the neurotransmitter levels in The System causing the desired response to be seen with a drug, substance or compound that is dependent on neurotransmitter for their effect, which displayed no response from initiation of use of a drug, substance, or compound that works with The System.

The balanced combination effectively increases the neurotransmitter levels in The System without causing depletion of neurotransmitters in one of the systems causing the desired optimal group response to be seen with a drug, substance or compound that is dependent on neurotransmitter for their effects, and displays a sub-optimal response from initiation of use or during use in a drug, substance or compound that works with The System. The balanced combination is effective in optimizing balanced neurotransmitter levels for treatment and relief of dysfunction as a treatment in and of itself in subjects suffering from dysfunction. Dysfunction relating to The System includes, but is not limited to, examples previously cited. It is known to the art of medicine that low levels of the neurotransmitters in the catecholamine system and/or serotonin system cause dysfunction.

The balanced combination is effective at treating dysfunction on a long-term basis. Long-term efficacy is a problem known to exist with use of drugs, compounds, or substances which exert effects on The System.

It is common in medicine to attribute some dysfunction to an imbalance of neurotransmitters. Applicant asserts that imbalance is no more than the fact that The System has lower levels than is required to prevent dysfunction. As noted previously, affecting change to one system will affect change in the other system. Even perceived imbalances in The System can be effectively managed with the use of the balanced combination.

The balanced combination is effective at inducing a display of new properties not previously known or seen in the past with neurotransmitter levels at or below the normal or reference range. In the normal diet with normal systems, tyrosine and tryptophan are synthesized into catecholamines and serotonin respectively. In the normal system, biochemical regulatory mechanisms exist; including enzymatic regulatory feed back, which limit the amount of catecholamines and serotonin synthesized into The System. The balanced combination can lead to a display of new clinical responses and properties that are not appreciated in a system with normal or low neurotransmitter levels. An example of this can be found where appetite suppression is observed with the use of this combination and Citalopram, where the caliber of appetite suppression observed is not seen with the components individually.

The balanced combination represents a component that can be used for elevating The System above levels normally found in the normal state. This elevated state can effectuate other clinical responses of The System and also with drugs, substances, or compounds that are dependent on neurotransmitters for their effects. It can give results not commonly associated with normal levels of the neurotransmitters in the catecholamine and/or serotonin system in treatment or when drugs, substances, or compounds, that are dependent on neurotransmitters for their effects, are used.

The benefits of balancing and optimizing neurotransmitter levels include:

1. Reestablishing a clinic response from a drug, substance, or compound that is dependent on neurotransmitters for their effects and has developed a tachyphylaxis and quit working.

2. Establishing a clinical response in circumstances where no clinical response is seen in subjects from initiation of the drug, substance or compound that is dependent on neurotransmitters for their effects.

3. Optimizing or enhancing the response from drugs, substances, and compounds that is dependent on neurotransmitters for their effects and work with the catecholamine system and/or serotonin system.

4. Optimizing neurotransmitter levels for treatment and relief of symptoms relating to dysfunction of The System.

5. Inducing a display of new properties not previously known or seen in the past as neurotransmitters of The System interact with The System in states of dysfunction or states altered by forces outside The System to include but not limited to alterations by drugs, substances, compounds, or organisms introduction them into The System.

5. Optimizing innervation, regulation, and function of other systems interacting with The System.

6. Establishing a side effect profile in use that is much lower than seen with use of individual amino acid components or amino acid components no in proper balance.

In the seven cases listed above, the mechanism of action for effective optimal group outcomes is the use of the balanced combination L-dopa and 5-HTP with adequate levels of cofactors leading to the balanced elevation of neurotransmitters of The System with neurotransmitter levels that are at or above normal levels in The System.

It is well known in medicine that low levels of neurotransmitters in one system or both systems of the catecholamine and serotonin systems are a primary cause of dysfunction. Examples of disease and sub-optimal function have been previously cited. It is a teaching of this invention that drugs, substances, and compounds that work by redistribution of neurotransmitters from one place to another such as from the vesicles of the axon terminal into the synapse or other sites outside the axon terminal work by effectively tricking the central and peripheral nervous systems into reacting as if it had more neurotransmitters in The System by redistribution of neurotransmitters. But the fact is there are no more neurotransmitters in the system. While effecting this redistribution, symptoms of dysfunction may be under control but as a whole there is not one additional molecule of neurotransmitters added to The System by the process. In the process, the neurotransmitter molecules have merely been redistributed and the low levels of neurotransmitters that existed in The System as a whole prior to redistribution are still present. With the redistribution of neurotransmitter molecules from the safety of the axon terminal vesicles where they are not subject to enzyme catalyzed COMT and MAO metabolism, further depletion of neurotransmitters of The System can occur if synthesis and metabolism are out of balance. A teaching of the invention is utilization of balanced amino acid precursors with adequate levels of cofactors can prevent further depletion of neurotransmitters of The System in such circumstances and keep The System function optimally in the group.

Side effects with the use of individual amino acid precursors or unbalanced amino acid combinations of the neurotransmitter discussed herein are known distract from treatment through intolerance of the amino acid being used leading to stopping of treatment. It is a teaching of this invention that with the use of properly balanced amino acids of both systems the side effects of the amino acid precursors of one system can be cancelled out or diminished by the side effects of the amino acids of the other system. Side effects rates as high as 70% have been observed in subjects under treatment with amino acids of the system that have not been properly balanced. It is an observation of this invention that the side effect profile, with the use of properly balanced amino acid precursor dosing, of both systems is similar to placebo as illustrated by the following data. Use of amino acid sthat are not properly balanced leads to marked increased in side effects leading to stopping treatment, decreased compliance with taking the amino acids properly, and a host of other things that distract from optimal results.

The side effect profile of balanced amino acids in treatment is as follows and shows a profile similar to placebo. Total number of visits with reported side effect is 100. N=1,604 total visits (6.23%), some subjects reported multiple side effects at these visits.

| | |
|---|---|
| 1. Dry mouth | 34 (2.1%) |
| 2. Insomnia | 14 (0.9%) |
| 3. Headache | 12 (0.7%) |
| 4. Nausea | 10 (0.6%) |
| 5. Dizziness | 6 (0.4%) |
| 6. Constipation | 6 (0.4%) |

The following side effects were reported at the rate of 0.2% or less (4 per 1,604 visits or less):

1. Moodiness (2)

2. Cold extremities (1)

3. Cravings (4)

4. Diarrhea (4)

5. Drowsy (2)

6. Irritability (2)

7. Fingers tingle (1)

8. Sweats (2)

9. Jittery (2)

10. Fatigue (4)

11. Flatulence (2)

12. Palpitations (4)

13. Flush face (1)

14. Hypoglycemia (1)

15. Light headed (2)

16. Sore tongue (glossitis) (4)

17. Depression (1)

18. Thirst (2)

19. Abdominal pain (1)

20. Abdominal burning (1)

21. Spots before eyes (1)

22. Non-specific dermatitis (2).

Bearing in mind the catecholamine pathway:

Tyrosine (or other precursors of L-dopa)→L-dopa→dopamine→norepinephrine→epinephrine

79% of subjects in the general population studied prior to treatment have low levels of epinephrine in the system as evidenced by urinary neurotransmitter testing. A goal of this invention is to optimize the entire catecholamine system with group treatment to include dopamine, norepinephrine, and epinephrine. Not all subjects ingesting L-dopa achieve normal levels of epinephrine. The following discussion concerns management of this problem.

Parkinsonism, is characterized by motor signs such as akinesia, rigidity (referred to medically as “cog-wheel rigidity”), and often tremor at rest (referred to medically as a “pill-rolling tremor”). The etiology of Parkinsonism is permanent system damage to the dopamine neurons of the substantia nigera in the central nervous system. A prototype relating to the development of Parkinsonism is the neurotoxic agent 1-methyl-4-phenyl-1,2,3,6-tetrahydropyridine (MPTP), which selectively induces permanent neuronal damage (apoptosis) to the dopaminergic neurons of the substantia nigera.

It is known in medicine that 3-Hydroxy-L-,t,tyrosine (hereafter referred to as “L-dopa”) is effective in ameliorating and controlling symptoms of Parkinsonism. L-dopa is currently the gold standard in medicine for the treatment of Parkinson's disease (PD) due to its outstanding initial clinical efficacy. Over time, systems under treatment with L-dopa in general need to have the daily dosing of L-dopa increased due to tachyphylaxis that develops in the course of treatment. With standard L-dopa/carbidopa treatment, there may be a need to increase L-dopa dosing in most subjects over time in order to maintain benefits of controlling symptoms of the disease. It is also known that carbidopa, in the combination of carbidopa and L-dopa, is effective in decreasing the average daily dosing of L-dopa needed in the treatment of Parkinson symptoms and in the process, decreases dose related side effects of L-dopa.

In long-term therapy (4 to 6 years) with L-dopa treatment, as a group, systems treated with L-dopa begin to suffer side effects from the L-dopa to include fluctuations, dyskinesias, toxicity, or loss of L-dopa efficacy. The etiology of these problems developing after long-term L-dopa therapy is believed to be due to neurotoxicity from L-dopa. The etiology of neurotoxicity from L-dopa is also believed to be due to depletion of s-adenosylmethionine (SAMe) by L-dopa therapy. It is known that methionine, dimethionine, and s-adenosylmethionine (SAMe) may prevent SAMe depletion and neurotoxicity, but little more is known about strategies in treating Parkinson and L-dopa neurotoxicity until a teaching of this invention.

Problems with current treatment approaches, which utilize the carbidopa/L-dopa combination, include:

1. Problems with the use of carbidopa in treatment include:

A. Carbidopa is a general decarboxylase inhibitor that does not cross the blood brain barrier and in the process decreases peripheral conversion of L-dopa to dopamine in first pass through the liver and other sites where an L-dopa active decarboxylase enzyme may be found peripherally.

B. Carbidopa in its peripheral systemic role as a general decarboxylase inhibitor also inhibits the conversion of 5-HTP to serotonin (5-HT) peripherally.

C. In consideration of the discussion of paragraphs 1.A and 1.B of this section, carbidopa use has significant effects on both components of The System peripherally, which are detrimental to optimal group system results.

D. A significant problem encountered with the use of carbidopa in treatment is long-term peripheral depletion of the catecholamine system to include dopamine, norepinephrine, and epinephrine, but the serotonin system as well, which in turn distracts from optimal results in addressing The System.

E. Peripheral side effects associated with carbidopa therapy can be due to long-term depletion of The System through inhibition of neurotransmitter synthesis.

2. Problem with the use of L-dopa in treatment includes, but are not limited to:

A. Tachyphylaxis with L-dopa in treatment is a known problem in treatment, leading to a need to increase dosing in order to achieve or continue the desired response and outcomes.

B. Administration of L-dopa depletes S-adenosylmethionine (SAMe), a critical methyl donor needed in numerous (42) major chemical pathways in the body.

C. L-dopa administration is associated with neurotoxicity, which is linked inducing SAMe depletion.

D. L-dopa may induce toxicity in dopamine neurons, due to catechol-autoxidation. Catechols are O-methylated by catechol-O-methyltransferase (COMT) in a SAMe consuming reaction, preventing the initiation of catechol autoxidation.

E. The many subjects under treatment with L-dopa for 4 to 6 years begin to suffer fluctuations, dyskinesias, toxicity, or loss of efficacy.

F. Administration of methionine, dimethionine, and S-adenosylmethionine are known to be protective against neurotoxicity, but no effective strategies have been developed in the use of these substances to optimize protection in order to facilitate optimal group outcomes of The System.

G. Cysteine with adequate levels of cofactors may be substituted for any of the three substances discuss in paragraph 2.F of this section (methionine, dimethionine, and S-adenosylmethionine) to optimize protection in order to facilitate optimal group outcomes of The System.

H. Depletion of SAMe is known to induce hyperhomocysteinemia with associated and known increase in risks associated with hyperhomocysteinemia.

I. It is known that hyperhomocysteinemia may be properly managed with the use of adequate amounts of vitamin B6, folic acid (folate) and vitamin B12.

J. Depletion of SAMe secondary to administration of L-dopa is known to be associated with depletion of glutathione leading to all known problems and risks associated with glutathione depletion. Glutathione depletion decreases ability to neutralize toxins in The System and its depletion enhances neurotoxicity of L-dopa in treatment.

K. Utilization of cysteine with cofactors will prevent glutathione depletion in L-dopa therapy.

L. Depletion of SAMe is known to compromise one carbon methylation though out the organism leading to a large variety of problems in which one carbon methylation by S-adenosylmethionine is needed for proper functioning of the biochemical system.

M. Depression is a known side effect of L-dopa therapy and is thought to be due to depletion of other neurotransmitters in The System during therapy by mechanisms discussed in this invention. Some of these neurotransmitters are dependent on proper levels of SAMe to be synthesized optimally.

N. L-dopa is used primarily in elderly humans for the treatment of Parkinsonism and depletion of SAMe by L-dopa can exacerbate Alzheimer's disease exacerbating cognitive function and other dysfunction of The System.

The invention manages these problems. Proper balanced use of vitamins, minerals and amino acids can prevent the problems encountered with L-dopa or L-dopa/carbidopa therapy.

It is commonly accepted that carbidopa can decrease the need for L-dopa in therapy of Parkinson patients in a when used in a 4:1 L-dopa:carbidopa ratio. This means that systems taking 400 mg of L-dopa per day to control symptoms in general may need only 100 mg of L-dopa per day to achieve the same response when carbidopa (in a 4:1 ratio of L-dopa:carbidopa dosing on a milligram per milligram basis) is co-administered and in the process, dose related side effects of L-dopa are decreased.

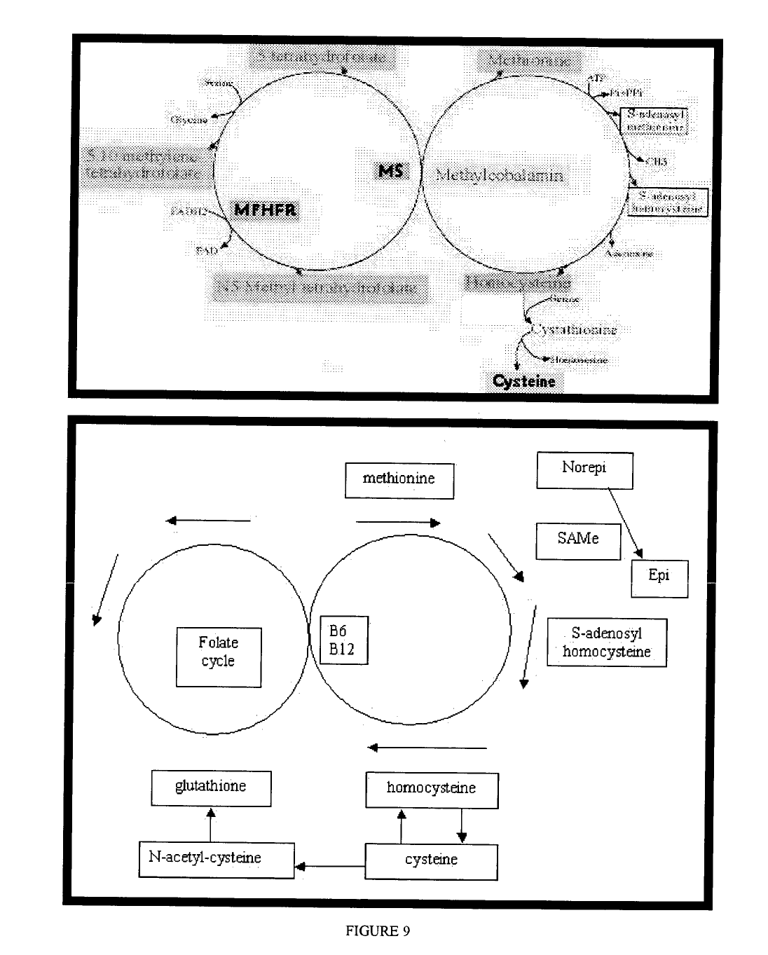

It is a known that L-dopa depletes SAMe significantly by mechanisms discussed and in the process all related systems dependant on one carbon methylation of SAMe are affected adversely. FIG. 9 is two drawings of the biochemical pathway involving SAMe, homocysteine, cysteine, N-acetyl-cysteine, glutathione, and methionine. In reviewing the pathways of FIG. 9, it is important to note that the rate limiting reaction of the right circular pathway is the conversion of homocysteine to methionine. In this reaction, it is known that deficiency in any of the following; vitamin B6, folate, and vitamin B12 leads to hyperhomocysteinemia and depletion of SAMe. This in turn causes dysfunction of The System (serotonin/catecholamine) as a whole since epinephrine synthesis which is dependent on SAMe is decreased leading to group system results that are not optimal in addressing dysfunction.

Once hyperhomocysteinemia develops, it is well-known that methionine and SAMe depletion occurs. As previously noted, depletion of SAMe by L-dopa is associated with development of hyperhomocysteinemia, the mechanism of this action is by removing sulfur based amino acid precursors from The System secondary to metabolism of the SAMe induced by L-dopa. It is known that the proper treatment of hyperhomocysteinemia involves administration of adequate amounts of vitamin B6, folate and B12 thus properly dealing with the hyperhomocysteinemia that occurs secondary to L-dopa therapy induced nutritional deficiency.

It has been recognized by applicant that when proper levels of vitamin B6, folate, and B12 are administered on a daily basis, it may take as long as three to six months to return the state of hyperhomocysteinemia to normal. In accordance with the invention, when treatment is initiated in a The System with low epinephrine levels with any of the following being deficient; SAMe, vitamin B6, folate, and vitamin B12 in proper amounts, it can take 3 to 6 months or more for the epinephrine levels to return to normal.

In referring to FIG. 9, cysteine is converted to homocysteine and can play a role as the sulfur donor amino acid that is the basis for the amino acids in right circular pathway of FIG. 9. Depletion of SAMe by L-dopa leads to depletion of the other sulfur bearing components of the right circular pathway of FIG. 9. It is known that depletion of SAMe leads to depletion of glutathione detoxification of The System toxin induced by L-dopa therapy. It is known in the literature that administration of methionine, dimethionine, and SAMe can prevent SAMe depletion, which is implicated in neurotoxicity from L-dopa therapy. It is known that administration of glutathione can increase SAMe levels. It is a teaching of this invention that cysteine may be substituted for methionine, dimethionine, and SAMe to prevent depletion.

Administration of cysteine with vitamin B6, folate, and vitamin B12 in proper dosing levels can also prevent the depletion of SAMe during L-dopa (or tyrosine) therapy when proper techniques and dosing is used as described herein. From an economic standpoint, use of cysteine to prevent SAMe depletion is much less expensive than SAMe, leading to more cost effective treatment. Proper dosing of cysteine is important in order to optimize group response. It has been found that a daily dosing of cysteine of 500 to 15,000 mg per day can be effective in subjects, but for optimal group treatment in healthy subjects, it is desirable to have a daily cysteine intake of 3,000 to 5,000 mg with preferred daily dosing for group treatment being 4,500 mg per day of cysteine based on the database supported research results leading up to this invention.

Cortisol synthesis is regulated in the following manner. Increased levels of norepinephrine stimulate corticotropin releasing factor (CRF). CRF in turn regulates Adrenalcorticotropic Hormone (ACTH). ACTH in turn regulated synthesis of cortisol. It is known that cortisol affects and stimulates synthesis of phenylethanolamine-N-methyltransferase (PNMT) at the DNA transcription level where even small increases in cortisol can be translated into a four or five fold increase in PNMT production. It is known that the enzyme phenylethanolamine-N-methyltransferase (PNMT) catalyses the conversion of norepinephrine to epinephrine. In this reaction, S-adenosyl methionine (SAMe) serves as a methyl donor in the conversion of norepinephrine to epinephrine. The use of vitamin B6, folate, vitamin B12, and cysteine optimizes SAMe synthesis. Two components and rate limiting factors in the synthesis of epinephrine are SAMe and cortisol. It is a teaching of this invention that by monitoring system epinephrine levels, with the preferred method being the epinephrine-creatinine ratio, optimal function of not just the SAMe system, but the cortisol system (synthesis of which is controlled by norepinephrine) as well can be monitored.

It is known that cortisol plays a key role in hormone regulation. Optimal hormone regulation can only be affected by optimizing the neurotransmitters of the catecholamine and serotonin system, which in turn by way of norepinephrine will optimize, regulate, and control the cortisol synthesis.

Administration of proper levels of cysteine (or methionine, dimethionine, SAMe,) or any other component of the right circular pathways in FIG. 9 can prevent SAMe depletion. The use of SAMe is not optimal in the presence of hyperhomocysteinemia, SAMe can exacerbate the hyperhomocysteinemia if proper dosing of vitamin B6, folate, and vitamin B12 is not in place, deficiencies of such being the primary cause of hyperhomocysteinemia. Optimal use of cysteine or other amino acid pathway components in the treatment of SAMe and glutathione depletion by L-dopa includes vitamin B6, folate, and vitamin B12 supplementation in appropriate levels.

Elevation of SAMe levels during L-dopa therapy via proper amino acid therapy with sulfur based amino acids and cofactors; protection is gained from the neurotoxic effects of L-dopa in treatment. It is a teaching of this invention that maintaining proper SAMe levels, the dosing needs for L-dopa decreases in treatment and tachyphylaxis is prevented in L-dopa therapy. Case studies indicate that dosing needs of L-dopa with optimized SAMe through proper use of amino acids containing the proper sulfur group outlined in FIG. 9 with proper cofactor administration are comparable or less than the needs of L-dopa in combination with carbidopa at initiation of treatment and in long term treatment less. As the SAMe levels are maintained with the proper use of cysteine and cofactors in the right circular pathway in FIG. 9 with vitamin B6, folate, and vitamin B12, tachyphylaxis and other side effects from L-dopa develop less frequently. In the files of this invention are case studies where subjects suffering from Parkinsonism symptoms who were taking 25 mg of carbidopa and 100 mg of L-dopa per day were switched to 120 mg of L-dopa with proper levels of cysteine with co-factors. In these case studies, subjects showed significant improvement in tremor and other symptoms over the L-dopa/carbidopa combination relating to Parkinsonism and Parkinson side effects in one to two weeks and experienced a lower rate of tachyphylaxis.

Proper use of cysteine (or the amino acid components of the right circular pathway of FIG. 9) with vitamins B6 and B12, and folate with balanced administration of L-dopa and 5-HTP with adequate levels of cofactors can eliminate the need for carbidopa in therapy, which in turn comprehensively manages the problems associated with L-dopa and/or carbidopa therapy.

Tyrosine administration on a long-term basis can also be associated with the problems cited with respect to the use of L-dopa. Table 1 is results of laboratory testing of two subjects who ingested 3,000 mg of tyrosine.

TABLE 1

| Time | Dopamine | | Norepinephrine | | Epinephrine | |
|---|---|---|---|---|---|---|
| (Minutes) | Subject 1 | Subject 2 | Subject 1 | Subject 2 | Subject 1 | Subject 2 |
| 0 | 5 | 48 | 1.3 | 2.0 | 3.3 | 5.7 |
| 30 | 1545 | 2780 | 18.2 | 2.1 | 31.8 | 15.5 |
| 45 | 39 | 1389 | 7.3 | 9.5 | 43.5 | 18.0 |
| 60 | 27 | 432 | 4.1 | 17.8 | 12.0 | 26.4 |
| 75 | 15 | 295 | 2.7 | 52.8 | 5.4 | 37.5 |
| 105 | 9 | 88 | 1.5 | 7.6 | 3.3 | 29.8 |
| 135 | 5 | 55 | 1.6 | 6.1 | 2.7 | 13.9 |
| 165 | 3 | 45 | 1.8 | 4.5 | 2.5 | 15.5 |

Table 1 illustrates the effects of ingestion of 3,000 mg of tyrosine on the urinary neurotransmitter-creatinine ratio as reported on the table in micrograms of neurotransmitters per gram of creatinine. The normal range for the laboratory methods used was previously reported in this invention. Clearly dopamine levels that are 6 to 11 times higher than the normal range are established for the dopamine levels 30 minutes after ingestion of tyrosine. This is evidence of the ability of tyrosine (or amino acid precursors of tyrosine that elevate tyrosine levels) to induce L-dopa levels higher than normal and the products of synthesis that it is involved in the face of norepinephrine-tyrosine hydroxylase regulation of catecholamine production. Systems ingesting tyrosine (or amino acid precursors of tyrosine) should be monitored for depletion of SAMe and glutathione, with all of the implications and considerations of L-dopa use as discussed applying, since tyrosine is the precursor of L-dopa. Table 1 clearly shows that marked elevations of dopamine and obvious implied elevations of its precursor L-dopa are possible with use of tyrosine by The System.

Tyrosine and tryptophan (or other amino acid precursors of dopamine or serotonin) can be substituted for L-dopa and 5-HTP respectively in the description of any teaching of this invention, but may lead to less than optimal group system results in those systems with a great enough disproportion between the electrical energy input and output of the neurons or neuron bundles secondary to biochemical enzyme regulation of tyrosine and tryptophan synthesis.

It is known that Citalopram is effective in treating bradykinesia that can be a problem or can develop during treatment with L-dopa or L-dopa combinations. Citalopram is known to be a highly specific serotonin reuptake inhibitor with very little activity in comparison to other selective re-uptake inhibitors on the catecholamine system. It is a teaching of this invention that this further demonstrates and reinforces that affecting a component of one system affects components of both systems. Here depletion of the catecholamine system and supporting components is compensated for by use of a drug that affects, in a highly selective manner, the serotonin system while the primary focus of treatment has been the catecholamine system.

L-dopa therapy impacts the serotonin system as discussed in FIG. 9. It is a teaching of this invention that proper use of 5-HTP with the appropriate cofactors has a positive impact on the side effect profile of L-dopa in therapy secondary to The System as a whole functioning optimally and the ability of 5-HTP to decrease the dosing needs of L-dopa needed to obtain the desired outcome of treatment thereby decreasing the rate of dose related side effects of L-dopa.

Depletion of SAMe by L-dopa during therapy affects all systems which are dependent on single carbon methylation by SAMe. By monitoring the products of synthesis involving one carbon methylation by SAMe properly outcomes can be optimized. Proper management of SAMe depletion is a teaching of this invention.

The preferred method for monitoring immediate products of synthesis involving SAMe methylation, but not limited to, is monitoring of the urinary epinephrine-creatinine ratios. If normal levels of epinephrine are present in testing the assumption can be made that the SAMe system is functioning optimally as evidenced by lab testing.

In the use of cysteine (or other sulfur based amino acid substances cited in FIG. 9 as previously discussed) with cofactors to prevent SAMe depletion, neurotoxicity, and other problems associated with L-dopa therapy, it is known in the literature that cysteine can concentrate methylmercury into the central nervous system leading to neurotoxicity. Selenium is known to bind to methylmercury, which in turn stabilizes it and renders it biologically inactive. Administration of cysteine, provisions should be made for co-administration of selenium or other substances capable of managing the methylmercury problems associated with cysteine administration to prevent neurotoxicity due to methylmercury concentrating into the central nervous system or other steps taken to insure that methylmercury toxicity is dealt with effectively.

In applications where less than all of the neurotransmitters of the catecholamine and serotonin systems are involved, even where only one of the neurotransmitters of The System is involved, optimal function of that application is only realized when all components of The System are functioning optimally. It is a teaching of this invention that only by the use of the balanced application of amino acids will The System function optimally and optimal group results be obtained, giving optimal results for the applications of the neurotransmitters where the catecholamine and serotonin systems are involved.

It is the goal of treatment to establish safe levels of neurotransmitters that are at or above the normal range as needed to successfully optimize The System and correct dysfunction. It is known that extremely high levels of neurotransmitters (100 to 3,000+times normal) are associated with disease and illness such as carcinoid syndrome, heart value disease, and other maladies. If huge amounts of L-dopa and 5-HTP are administered under conditions where ample cofactors and active enzymes are available, huge amounts of catecholamines and serotonin respectively are synthesized into The System since synthesis in both systems is not regulated by a biochemical enzymatic regulation.

U.S. Pat. Nos. 6,384,088 and 6,403,657 relate to treatment of obesity. It is a teaching of this invention that only by eating less calories on a day-to-day basis can systems suffering from obesity be relieved of the obesity. Furthermore, only by getting the appetite under control, can eating less food be affected comfortably. The only drugs known in medicine that affects appetite suppression is those drugs that work with the serotonin and/or norepinephrine systems. In order to get appetite under control to address obesity of the organism, you must control these systems. The methods of this invention can be used effectively to address the problem of obesity in the organism.

The following is a general discussion of management of considerations that may distract from optimal group outcomes in the use of amino acid to treat dysfunction. GI upset, including nausea, is a problem encountered with amino acid therapy that may lead to stopping treatment and in the process contributes greatly to decreased group results if this happens. GI upset is divided into two groups, “start up” and “carbohydrate intolerance”. It would appear that the problem in the past had not been fully understood. Once the cause of these problems are understood, they are easily managed, giving subjects the full ability to use amino acid therapy in the treatment of disease. About 1 of every 200 subjects experience GI upset on starting treatment. This GI upset typically builds with every dose of amino acids until about 3 days into treatment the subject can no longer tolerate symptoms and stops the amino acids. Database research in the past has shown that subjects who experience this problem in general, are the most neurotransmitter-depleted subjects as evident by the large number of other dysfunction related problems present. It is ironic that these rare subjects are the ones that need amino acids the most for neurotransmitter dysfunction. Subjects should be warned about this problem at initiation of therapy. The problem is best managed by restarting the patient on a very low dose at bedtime (when ready to go to sleep). Then increase the dosing only after 3 to 4 days of no symptoms, with subsequent increases in dosing in a similar manner until the normal starting dose is achieved in 3 to 4 weeks.

Up to 70% of subjects reported periodic GI upset after treatment has been in place for many days or weeks. The cause of the GI upset is not the amino acids but a carbohydrate intolerance that had developed with treatment. Carbohydrates are high calorie food with very little nutritional value. Common examples include, bread, noodles, candy, cereals, chips, popcorn, pies, cakes, pop, pancakes, waffles, and syrup just to name a few. Typically, intolerance symptoms come on 2 or 3 hours after eating and last 30 minutes to an hour. Changing one food in the diet usually is all that is needed. For example, we have seen subjects who changed from white bread to whole wheat bread and no longer experience further symptoms.

2. Description of the Preferred Embodiment.

In general, the preferred embodiment of the therapy and therapy of the present invention involves the use or administration of the amino acids L-dopa and 5-HTP to increase levels of neurotransmitters of The System uniformly throughout the body.

Therapeutic daily dosing ranges of L-dopa and 5-HTP are:

1. L-dopa, 5 mg to 3,000 mg per day

2. 5-HTP, 10 mg to 2,000 mg per day

The primary amino acid combination of 5-HTP and L-dopa as disclosed above should, preferably, be supported by the use of cofactors in the following daily dosing range:

1. Vitamin B6, 2 mg to 300 mg per day.

2. Vitamin C, 50 mg to 2,000 mg per day.

3. Calcium, 50 mg to 2,000 mg per day.

4. Folate (folic acid), 50 mg to 4,000 mg per day.

5. Cysteine, 100 mg to 15,000 mg per day.

Tyrosine and tryptophan may be substituted for L-dopa and 5-HTP respectively in treatment of subjects where the mechanism of dysfunction does not require establishment of neurotransmitter levels significantly above the reference or normal range in order to facilitate optimal group results. The following are preferred daily dosing ranges with respect to the substitutions:

1. Tyrosine, 100 mg to 9,000 mg per day.

2. Tryptophan, 50 mg to 9,000 mg per day.

The following option is available in the form of substituting other amino acid precursors for tyrosine, on a daily dose basis:

1. D.L-Phenylalanine or the active isomer thereof, 10 mg to 6,000 mg per day.

2. N-acethyl-L-tyrosine, 10 mg to 6,000 mg per day.

3. Any other amino acid or amino acid precursor or amino acid intermediate of dopamine in mg. amounts equivalent to dosing of items 1 and 2 above.

For optimal results, the amino acid combinations should be administered under the care of a trained caregiver. The preferred method of administration is orally in pill or a powdered form that may be mixed with water based flavored liquid. Intranasal spray is an option.

The pharmacological formula and therapy disclosed above functions by increasing neurotransmitter levels uniformly, in a balanced manner, of the catecholamine system and the serotonin system. A steady state is generally achieved in five to seven days once the combination is started or a change in the dosing of the combination has occurred, provided that there has not been a significant dietary change.

The formula and therapy of the present invention, involving balanced elevation of the neurotransmitters in support of applications where elevations of the catecholamine and/or serotonin system are desirable, may be applied in situations including, but not limited to:

1. Reestablishing a clinic response from a drug, substance or compound that is dependent on neurotransmitters for their effects, that has developed a tachyphylaxis and quit working.

2. Establishing a response in circumstances where no response is seen in subjects from initiation of the drug, substance, or compound that is dependent on neurotransmitters for their effects.

3. Optimizing or enhancing the response from drugs, substances, or compounds that is dependent on neurotransmitters for their effects that work with The System.

4. Optimizing neurotransmitter levels for treatment and relief of symptoms relating to The System disease.

5. Inducing a display of new properties not previously known or seen in the past with balanced neurotransmitter of The System levels at or below the normal range.

1. Any application where any neurotransmitter component of The System affects, regulates, or controls outcomes in order to optimize the outcome.

2. In situations where unbalanced use of amino acids previously has induced a state where side effects caused discontinuation of treatment.

3. Any event where The System interacts or is required.

Table 2 specifies a preferred dosing schedule for combinations D5 and D6.

TABLE 2
Dosing schedule for adjusting D5 and D6.

| | | | | 2 to 3 hours |
|---|---|---|---|---|
| | AM | Noon | PM | before bed. |
| Step 1/week 1 | ½ daily dose | — | ½ daily dose | — |
| | D5 | | D5 | |
| Step 2/week 2 | ½ daily dose | ½ daily dose | One daily | — |
| | D5 | D5 | dosing D6 | |
| Step 3/week 3 | ½ daily dose | ½ daily dose | One daily | One daily |
| | D5 | D5 | dosing D6 | dosing D6 |
| Step 4/week 4 | ½ daily dose | ½ daily dose | One daily | One daily |
| | D5 and ½ daily | D5 and ½ daily | dosing D6 | dosing D6 |
| | dosing D6 | dosing D6 | | |
| Step 5/week 5 | ½ daily dose | ½ daily dose | 1.5 daily dosing | 1.5 daily dosing |
| | D5 and ½ daily | D5 and ½ daily | D6 | D6 |
| | dosing D6 | dosing D6 | | |
| Step 6/week 6 | ½ daily dose | ½ daily dose | 2.0 daily dosing | 2.0 daily dosing |
| | D5 and ½ daily | D5 and ½ daily | D6 | D6 |
| | dosing D6 | dosing D6 | | |

Dosing times are:
AM = when the subject gets up.
Noon = 4 to 5 hours after the subject gets up.
PM = 8 to 10 hours after the subject gets up.

The preferred combination referred to as “D5” contains the following daily dosing components:

1. 5-HTP, 300 mg.

2. L-dopa, 120 mg.

3. Vitamin B6, 75 mg.

4. Vitamin C, 1,000 mg.

5. Calcium (preferably in the form of calcium citrate) 1,000 mg.

6. Folate 400 mcg.

7. L-Lysine, 500 mg.

An additional formulation combination, referred to as “D6” and “a full dose of D6” contains the following dosing components:

1. 5-HTP, 300 mg.

2. L-dopa, 60 mg.

There are a number of dosing possibilities with the formulas. The goal of treatment should be to establish an initial dosing level with subsequent increases in dosing where virtually all subjects receive optimal results within 4 to 5 weeks of starting treatment. By following the preferred dosing schedule of Table 2, the treatment goal will be facilitated. This facilitates optimal group results in that if dosing levels of amino acids are started at too low a rate and increased too slowly it might take months to optimize some subjects and see the desired results during which time subjects are prone to drop out of treatment secondary to not achieving relief of symptoms affecting optimal results of the group greatly.

On day one of treatment, the subject is started on D5, which is divided into two equal daily doses given in the AM or when the subject gets up and approximately 4 or 5 PM in the afternoon or 8 to 10 hours after getting up. If the subject, on the seventh day after treatment was started, is not experiencing control of dysfunction, the subject should have D5 and D6 adjusted as follows. The subject should be instructed to take 1/2 daily dosing of D5 in the AM or when the subject gets up and 1/2 daily dosing of D5 one hour before the noon meal or 4 to 5 hours after getting up. D6 should be added as a full dosing approximately 4 to 5 PM or 8 to 10 hours after getting up. This procedure is repeated under the dosing schedule outlined in Table 2 until optimal neurotransmitter levels are established as evidenced by resolution of symptoms of dysfunction, at which point, the system is optimized.

In some cases of extreme dysfunction it may be desirable to achieve neurotransmitter levels greater than these. It is noted that cases may exist such as end stage Parkinsonism where dosing higher than the therapeutic range is needed to control symptoms of dysfunction.

The initial dosing of D5 in divided doses twice a day need never to be increased. Additional adjustment of the combination should be affected with D6. The reason for this is that on a daily basis subject do not need the additional cofactors required for optimal function that is found in the D5 and D6. The dosing of the combination is adjusted after the first week by adding a daily dosing of D6 of Table 2 each week until the desired response is seen according to the dosing schedule as described for day 8 of this section. The majority of subjects will not need dosing higher than step 3/week 3.

At the start of treatment where L-dopa and/or tyrosine (or other amino acid precursors of dopamine) are components of the therapy, the following components should be started:

1. Cysteine in the amount of 1,500 mg three times a day for a total daily dosing of 4,500 mg per day in order to prevent depletion of SAMe-glutathione systems as covered and illustrated in FIG. 9.

2. Selenium in the amount of 400 mcg per day in divided dosing of 133⅓ mcg three times a day should be given to prevent neurotoxicity during administration of cysteine.

3. Folate in the amount of 400 mcg per day in divided doses of 133⅓ mcg three times a day to facilitate proper function of the homocysteine-methionine-SAMe cycle as illustrated in FIG. 9. It is noted that folate may mask megaloblactic processes.

4. Vitamin B6 in the amount of 75 mg per day in divided doses of 25 mg three times a day to facilitate proper function of the homocysteine-methionine-SAMe cycle as illustrated in FIG. 9. Vitamin B6 is also a cofactor in the synthesis of catecholamines (dopamine, norepinephrine, and epinephrine) as well as serotonin.

5. Vitamin B12 in the amount of 10 mcg per day in divided doses of 3⅓ mg three times a day to facilitate proper function of the homocysteine-methionine-SAMe cycle as illustrated in FIG. 9 in people with megaloblastic disease or at the discretion of the caregiver. It is noted that ingestion of folate may mask megaloblastic disease and consideration should be made.

6. 5-hydroxytrptophan (5-HTP) in the amount of 300 mg per day in divided doses of 100 mg three times a day. It is known that administration of L-dopa depletes serotonin and administration of 5-HTP prevents depletion of serotonin.

Treatment may continue for prolonged periods of time, including up to lifetime, if needed.

Both the catecholamine system and the serotonin systems must be addressed properly for optimal treatment of a group to be affected. Addressing only one neurotransmitter or neurotransmitter system such as the catecholamine system or the serotonin system with drugs, amino acids, or other means that affect the relative levels of neurotransmitters will not give optimal group results in the treatment of The System afflicted with neurotransmitter “dysfunction”.

No new side effects are associated with use of the invention other than those symptoms seen with use of the individual components of the invention. Side effects of higher dose amino acid there associated with administration of the individual components cited in the invention are lower then the amino acids cited in the invention are given in combination. An additional degree of safety is provided due to the low dose of L-dopa most systems require for treatment of dysfunction to include Parkinsonism treatment under this invention, being approximately 1/4 the standard starting dose of L-dopa when used alone in the treatment Parkinson disease in medicine.

The use of catecholamine and serotonin amino acid precursors in proper balance off sets and cancels side effects seen in use of one system alone. This makes treatment more tolerable and thereby enhances outcomes of group treatment. Proper ratios for administering amino acid precursors of dopamine and serotonin are as follows with dosing changes within 85% of the stated values on a milligram per milligram basis having some benefit in minimizing side effects encountered in use of unopposed amino acid precursors of one system when used singularly.

| 1. Tyrosine to 5-HTP ratio | 10:1 on a milligram basis |
|---|---|
| 2. L-dopa to 5-HTP | 3:1 |
| 3. Phenylalanine to 5-HTP | 10:1 |
| 4. N-acetyl-tyrosine to 5-HTP | 5:1 |

5. Other amino acid precursors of serotonin and dopamine may be
used as long as proper consideration is given for optimal ratio
dosing.

The descriptions above and the accompanying drawings should be interpreted in the illustrative and not the limited sense. While the invention has been disclosed in connection with an embodiment or embodiments thereof, it should be understood by those skilled in the art that there may be other embodiments which fall within the scope of the invention as defined by the claims. Where a claim, if any, is expressed as a means or step for performing a specified function it is intended that such claim be construed to cover the corresponding structure, material, or acts described in the specification and equivalents thereof, including both structural equivalents and equivalent structures, material-based equivalents and equivalent materials, and act-based equivalents and equivalent acts.

What is claimed is:

1. A method of treating a patient comprising the steps of:
a. administering an amino acid precursor of a catecholamine; and
b. administering an amino acid precursor of serotonin.

2. The method of claim 1, wherein the precursor of serotonin and the precursor of catecholamine are provided in an effective therapeutic range to control dysfunction.

3. The method of claim 2, wherein the effective therapeutic range comprises:
a. 5-9000 mg per day of the precursor of a catecholamine; and
b. 10-9000 mg per day of the precursor of serotonin.

4. The method of claim 3, wherein the catecholamine is dopamine.

5. The method of claim 4, wherein the precursor of dopamine is selected from the group of amino acids consisting of L-dopa, tyrosine, D,L-Phenylalanine or an active isomer thereof, and N-acetyl-L-tyrosine.

6. The method of claim 5, wherein the precursor of dopamine is L-dopa, and wherein the effective therapeutic range thereof is 5-3000 mg per day.

7. The method of claim 5, wherein the precursor of dopamine is tyrosine, and wherein the effective therapeutic range thereof is 100-9000 mg per day.

8. The method of claim 5, wherein the precursor of dopamine is D,L-Phenylalanine or an active isomer thereof, and wherein the effective therapeutic range thereof is 10-6000 mg per day.

9. The method of claim 5, wherein the precursor of dopamine is N-acetyl-L-tyrosine, and wherein the effective therapeutic range thereof is 10-6000 mg per day.

10. The method of claim 3, wherein the precursor of serotonin is selected from the group of amino acids consisting of 5-HTP and tryptophan.

11. The method of claim 10, wherein the precursor of serotonin is 5-HTP, and wherein the effective therapeutic range thereof is 10-2000 mg per day.

12. The method of claim 10, wherein the precursor of serotonin is tryptophan, and wherein the effective therapeutic range thereof is 50-9000 mg per day.

13. The method of claim 1, further comprising the step of administering at least one cofactor, selected from the group of cofactors consisting of vitamin B6, Vitamin C, Calcium, Folate, and Cysteine.

14. The method claim 1, further comprising the step of administering at least one cofactor, selected from the group of cofactors consisting of vitamin B6 in an effective therapeutic range of 2-300 mg per day, Vitamin C in an effective therapeutic range of 50-2000 mg per day, Calcium in an effective therapeutic range of 50-2000 mg per day, Folate in an effective therapeutic range of 50-4000 mcg per day, and Cysteine in an effective therapeutic range of 100-15000 mg per day.

15. The method of claim 14, wherein all of the cofactors are administered.

16. The method of claim 1, wherein:
the precursor of a catecholamine is L-dopa administered in an amount of 120 mg per day; and
the precursor of serotonin is 5-HTP administered in an amount of 300 mg per day;
and further comprising the step of administering cofactors:
Vitamin B6 in amount of 75 mg per day,
Vitamin C in an amount of 1000 mg per day,
Calcium in an amount of 1000 per day, and
Folate in an amount of 400 mcg per day.

17. The method of claim 16, wherein the combination of precursors and cofactors is administered daily for at least eight days.

18. The method of claim 17, wherein the combination of precursors and cofactors is administered daily, a first half of the specified amounts being administered approximately at a morning meal and the second half of the specified amounts being administered approximately at an evening meal.

19. The method of claim 18, further comprising the step of examining the patient to determine whether control of dysfunction has been attained seven days after treatment has been initiated, and if control of dysfunction has not been attained, on at least the seventh day after treatment has been initiated administering the second half of the specified amount of the combination at a noon meal, and administering a second combination of precursors daily at approximately the evening meal, the second combination comprising:
L-dopa at an amount of 60 mg, and
5-HTP at an amount of 300 mg.

20. The method of claim 1, further comprising the step of administering Selenium in the amount of 400 mcg per day.

21. The method of claim 1, further comprising the step of administering Vitamin B12 in the amount of 10 mcg per day.

22. The method of claim 2, wherein the effective therapeutic range comprises:
a. from 3-10 parts per day of the precursor of a catecholamine; and
b. 1 part per day of the precursor of serotonin.

23. The method of claim 22, wherein the precursor of serotonin is 5-HTP.

24. The method of claim 23, wherein the precursor of catecholamine is L-dopa provided in 3 parts per day.

25. The method of claim 23, wherein the precursor of catecholamine is N-acetyl-L-tyrosine provided in 5 parts per day.

26. The method of claim 23, wherein the precursor of catecholamine is tyrosine provided in 10 parts per day.

27. The method of claim 23, wherein the precursor of catecholamine is D,L-Phenylalanine or an active isomer thereof provided in 10 parts per day.

28. A method of treating neurotransmitter dysfunction in a patient comprising the steps of:
a. administering an amino acid precursor of a catecholamine in an effective therapeutic range, the catecholamine precursor being selected from the group of amino acids consisting of L-dopa, tyrosine, D,L-Phenylalanine or an active isomer thereof, and N-acetyl-L-tyrosine;
b. administering an amino acid precursor of serotonin in an effective therapeutic range, the serotonin precursor being selected from the group of amino acids consisting of 5-HTP and tryptophan; and
c. administering at least one cofactor, selected from the group of cofactors consisting of vitamin B6, Vitamin C, Calcium, Folate, and Cysteine.

29. A method of treating dysfunction in the serotonin and catecholamine neurotransmitter system in a patient comprising the steps of:
a. administering, daily, a first combination of components for at least eight days, the first combination comprising:
i. an amino acid precursor of a catecholamine component in an effective therapeutic amount of approximately 120 mg, the catecholamine precursor being selected from the group of amino acids consisting of L-dopa, tyrosine, D,L-Phenylalanine or an active isomer thereof, and N-acetyl-L-tyrosine;
ii. an amino acid precursor of serotonin component in an effective therapeutic amount of approximately 300 mg, the serotonin precursor being selected from the group of amino acids consisting of 5-HTP and tryptophan; and
iii. a cofactor component, the cofactor component comprising at least one subcomponent selected from the group of cofactor subcomponents consisting of vitamin B6, Vitamin C, Calcium, Folate, and Cysteine;
a first half, with respect to quantity, of the first combination being administered approximately at a morning meal, and the second half, with respect to quantity, of the first combination being administered at least approximately four hours after administration of the first half of the first combination;
b. on the seventh day after treatment has been initiated, determining whether dysfunction in the patient has been controlled; and
c. if, in step b, dysfunction has not been controlled, administering, daily, a second combination of components comprising:
i. an amino acid precursor of a catecholamine component in an effective therapeutic amount of approximately 60 mg, the catecholamine precursor being selected from the group of amino acids consisting of L-dopa, tyrosine, D,L-Phenylalanine or an active isomer thereof, and N-acetyl-L-tyrosine; and
ii. an amino acid precursor of serotonin component in an effective therapeutic amount of approximately 300 mg, the serotonin precursor being selected from the group of amino acids consisting of 5-HTP and tryptophan;
the second combination being administered at least approximately four hours after administering the second half of the first combination.
